(19) United States
(12) Patent Application Publication
(10) Pub. No.: US 2014/0303499 A1
(43) Pub. Date: Oct. 9, 2014

(54) ULTRASOUND DIAGNOSTIC APPARATUS AND METHOD FOR CONTROLLING THE SAME

(71) Applicant: KONICA MINOLTA, INC., Tokyo (JP)

(72) Inventors: Tadamasa TOMA, Osaka (JP); Jun OHMIYA, Kyotanabe-shi (JP); Bumpei TOJI, Hashima-shi (JP)

(73) Assignee: KONICA MINOLTA, INC., Tokyo (JP)

(21) Appl. No.: 14/246,918

(22) Filed: Apr. 7, 2014

(30) Foreign Application Priority Data
Apr. 9, 2013 (JP) 2013-080941

(51) Int. Cl. A61B 8/06; A61B 8/08

(52) U.S. Cl. CPC A61B 8/06; A61B 8/488; A61B 8/08
USPC 600/454

(57) ABSTRACT

An ultrasound diagnostic apparatus measures blood flow velocity by emitting ultrasound towards a measurement target and receiving a reflected wave, via an ultrasound probe. The ultrasound diagnostic apparatus includes an ultrasound image acquisition unit, a vascular region detection unit, a measurement position determination unit, and a Doppler gate setting unit. The ultrasound image acquisition unit acquires an ultrasound image which has been captured of the measurement target. The vascular region detection unit detects, from the ultrasound image, a vascular region corresponding to a blood vessel. The measurement position determination unit detects a specific part of the vascular region based on vascular region shape and determines, in accordance with the specific part, one or more measurement positions in the vascular region in terms of longitudinal direction thereof. The Doppler gate setting unit sets, with respect to each measurement position, a Doppler gate in a lumen region of the vascular region.

This application is based on an application No. 2013-080941 filed in Japan on Apr. 9, 2013, the contents of which are hereby incorporated by reference.

### BACKGROUND OF THE INVENTION

(1) Field of the Invention

The present invention relates to an ultrasound diagnostic apparatus, and a control method thereof, that automatically sets a measurement position for Doppler ultrasound based on results of ultrasound image analysis.

(2) Description of the Related Art

Diagnostic imaging apparatuses which are commonly used on living organisms include X-ray diagnostic apparatuses, MRI (Magnetic Resonance Imaging) diagnostic apparatuses and ultrasound diagnostic apparatuses. Among the above apparatuses, ultrasound diagnostic apparatuses in particular have advantages in terms of non-invasiveness and real time usage, and are widely used in health examinations and diagnosis.

An ultrasound diagnostic apparatus may use various different measurement methods. In brightness mode (B-mode) ultrasound, a cross-sectional image is acquired of a plane along which ultrasound travels. In motion mode (M-mode) ultrasound, movement perpendicular to a direction in which ultrasound travels is detected. In Doppler ultrasound, movement parallel to the direction in which ultrasound travels is detected. Measurement by Doppler ultrasound requires accurate setting of a Doppler gate at a position suitable for measurement. For example, when a stenosis is present in a blood vessel, the Doppler gate should be set at a position in the blood vessel at which the blood vessel in narrowest (Standard method for ultrasound evaluation of carotid artery lesions; Japan Journal of Medical Ultrasonics; 502; Vol. 36; No. 4; 2009).

### SUMMARY OF THE INVENTION

### Problems to be Solved by the Invention

Unfortunately, blood flow velocity differs at a position corresponding to the stenosis compared to a position separated from the stenosis. As a result, a doctor or other operator is required to possess a high degree of procedural skill in order to accurately set the Doppler gate at the position corresponding to the stenosis.

In consideration of the problem described above, the present invention aims to provide an ultrasound diagnostic apparatus, and a control method thereof, that enables setting of a Doppler gate at a position which is appropriate for measuring blood flow velocity.

### Means for Solving the Problems

One aspect of the present invention is an ultrasound diagnostic apparatus for measuring blood flow velocity by emitting ultrasound towards a measurement target via an ultrasound probe and by receiving a reflected wave via the ultrasound probe, the ultrasound diagnostic apparatus comprising: an ultrasound image acquisition unit configured to acquire an ultrasound image which has been captured of the measurement target; a vascular region detection unit configured to detect, from the ultrasound image, a vascular region corresponding to a blood vessel in the measurement target; a measurement position determination unit configured to detect a specific part of the vascular region based on shape of the vascular region and to determine, in accordance with the specific part, one or more measurement positions in the vascular region in terms of longitudinal direction thereof; and a Doppler gate setting unit configured to set, with respect to each of the measurement positions, a Doppler gate in a lumen region of the vascular region.

### Advantageous Effect of the Invention

The ultrasound diagnostic apparatus relating to the one aspect of the present invention detects the specific part, which for example corresponds to a diseased part or a diagnostic target in the measurement target, based on shape of the vascular region, automatically determines the one or more measurement positions in terms of the longitudinal direction of the blood vessel, and sets an appropriate Doppler gate with respect to each of the measurement positions. Therefore, the ultrasound diagnostic apparatus relating to the one aspect of the present invention enables simple and accurate measurement of blood flow velocity.

### BRIEF DESCRIPTION OF THE DRAWINGS

These and the other objects, advantages and features of the invention will become apparent from the following description thereof taken in conjunction with the accompanying drawings which illustrate a specific embodiment of the invention.

In the drawings:

FIG. 1 is a block diagram illustrating configuration of an ultrasound diagnostic apparatus 10;

FIG. 2A illustrates an example of determining a measurement position in the ultrasound diagnostic apparatus 10, FIG. 2B illustrates an example of setting a Doppler gate corresponding to the measurement position in the ultrasound diagnostic apparatus 10, and FIG. 2C illustrates an example of setting width of the Doppler gate in the ultrasound diagnostic apparatus 10;

FIG. 3 is a flowchart illustrating operation of the ultrasound diagnostic apparatus 10;

FIG. 4 illustrates an example of determining a plurality of measurement positions in the ultrasound diagnostic apparatus 10;

FIG. 5 is a flowchart illustrating operation of the ultrasound diagnostic apparatus 10 as relating to supplementary explanation (9) of a first embodiment;

FIG. 6 illustrates an example of segmentation and determination of measurement positions relating to a modified example of the first embodiment;

FIG. 7 is a block diagram illustrating configuration of an ultrasound diagnostic apparatus 20;

FIG. 8A illustrates an example of constructing a three dimensional (3D) image of a carotid artery, FIG. 8B illustrates an example of acquiring a current position and current orientation of an ultrasound probe, and FIG. 8C illustrates relationship between the 3D image and a scan plane of the ultrasound probe;

FIG. 9 is a flowchart illustrating operation of the ultrasound diagnostic apparatus 20;

FIG. 10 is a block diagram illustrating configuration of an ultrasound diagnostic apparatus 30;

FIG. 11A illustrates an example of segmentation and determination of measurement positions for a carotid artery, and FIG. 11B illustrates relationship between a plaque and results of blood flow velocity measurement in the carotid artery;

FIG. 12 is a flowchart illustrating operation of the ultrasound diagnostic apparatus 30;

FIGS. 13A-13C illustrate a situation in which a control method for an ultrasound diagnostic apparatus is implemented by a computer system, using a program stored on a recording medium such as a floppy disk;

FIG. 14A is an overview diagram of an ultrasound probe and a scan plane, FIG. 14B illustrates a method for scanning a carotid artery, FIG. 14C is a cross-sectional image along a longitudinal direction of the carotid artery, and FIG. 14D is a cross-sectional image along a transverse direction of the carotid artery;

FIG. 15A illustrates structure of a vascular wall of an artery in the transverse direction, FIG. 15B illustrates relationship between the vascular wall and a cross-sectional image of the artery in the transverse direction, FIG. 15C illustrates structure of the vascular wall of the artery in the longitudinal direction, and FIG. 15D illustrates a plaque in a cross-sectional image of the artery in the longitudinal direction;

FIG. 16 illustrates parts of a carotid artery;

FIG. 17 illustrates an example of determining a measurement position in a conventional ultrasound diagnostic apparatus;

FIG. 18A illustrates correspondence between a carotid artery and transverse cross-sectional images, FIG. 18B illustrates detection of a center point of a double loop in a transverse cross-sectional image, FIG. 18C illustrates a method of calculating intima-media thickness (IMT) in the transverse cross-sectional image, and FIG. 18D illustrates results of IMT calculation in the transverse cross-sectional image;

FIG. 19A illustrates setting of a Doppler gate in a transverse cross-sectional image, and FIG. 19B illustrates positional relationship of the Doppler gate and an ultrasound probe;

FIG. 20A illustrates configuration of a 3D model of a carotid artery and an ultrasound probe, and FIG. 20B illustrates an assistance image provided in order to guide the ultrasound probe;

FIG. 21 is a flowchart illustrating operation for setting a Doppler gate by generating a 3D model from a plurality of transverse cross-sectional images;

FIG. 22 illustrates correspondence between a plurality of transverse cross-sectional images and segmentation of a carotid artery;

FIG. 23 is a flowchart illustrating operation for analyzing a plurality of transverse cross-sectional images and for performing segmentation in accordance with structure of a carotid artery; and

FIG. 24 is a flowchart illustrating operation for analyzing a plurality of transverse cross-sectional images and for performing a finer degree of segmentation in accordance with whether a plaque is present.

### DESCRIPTION OF THE PREFERRED EMBODIMENTS

### Background Leading to Embodiments of the Invention

An ultrasound diagnostic apparatus may be used on various diagnostic targets such as blood vessels, heart, liver and breasts. In particular, in recent years there has been interest in using examination of the carotid artery in order to determine risk of arteriosclerosis.

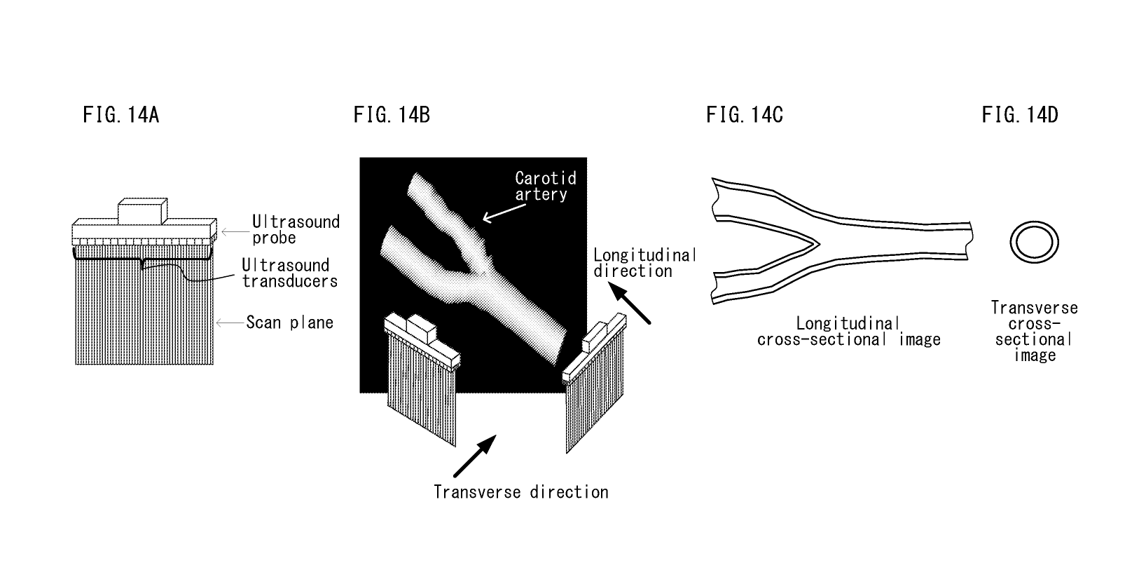

The following explains examination of the carotid artery using ultrasound. FIGS. 14A-14C are provided in order to explain image appearance when the carotid artery is scanned using ultrasound. FIG. 14A illustrates an ultrasound probe and a scan plane of the ultrasound probe. Ultrasound transducers are arranged in the ultrasound probe. When the ultrasound transducers are in a one dimensional (1D) array, as in the present example, a reception signal is acquired for a two dimensional (2D) scan plane directly below the ultrasound transducers, and a B-mode ultrasound image is acquired which is a cross-sectional image. As illustrated in FIG. 14B, during examination of the carotid artery, typically images are acquired in both a direction in which the carotid artery extends (herein referred to as a longitudinal direction), and also in a direction which is roughly perpendicular to both the longitudinal direction and a depth direction of the skin (herein referred to as a transverse direction). When the ultrasound probe is scanned in the transverse direction along the carotid artery, a cross-sectional image along a longitudinal axis of the carotid artery is acquired (herein referred to as a longitudinal cross-sectional image), such as illustrated in FIG. 14C. On the other hand, when the ultrasound probe is scanned in the longitudinal direction along the carotid artery, a cross-sectional image cutting across the carotid artery in the transverse direction is acquired (herein referred to as a transverse cross-sectional image), such as illustrated in FIG. 14D.

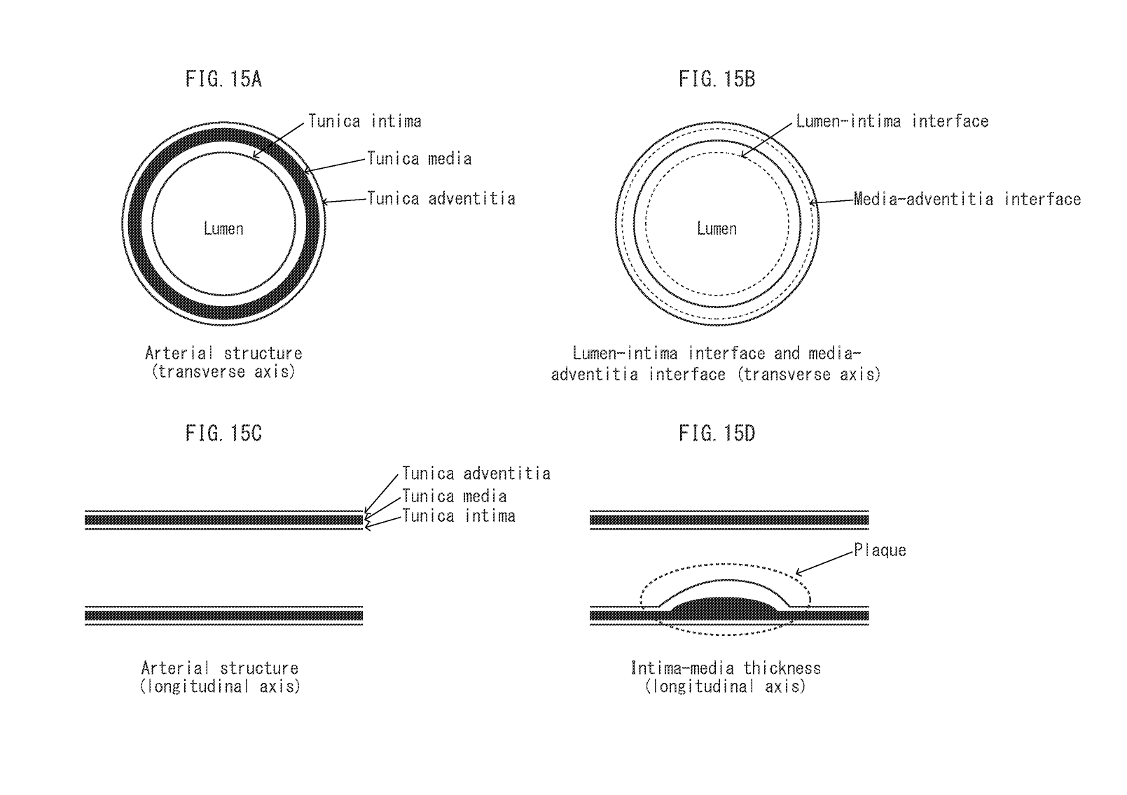

In examination of the carotid artery, vascular wall thickness of the carotid artery is used as an indicator of a degree of progression of arteriosclerosis. The following explains vascular wall structure of an artery with reference to FIGS. 15A-15C. The vascular wall of the artery has a three layer structure composed of a tunica intima, a tunica media, and a tunica adventitia. FIG. 15A illustrates structure of the vascular wall in the transverse direction, and FIG. 15C illustrates structure of the vascular wall in the longitudinal direction. Progression of arteriosclerosis is accompanied by hypertrophy of mainly the tunica intima and the tunica media. Consequently, in examination of the carotid artery using ultrasound, thickness of an intima-media complex composed of the tunica intima and the tunica media (herein referred to as IMT) is measured by detecting an interface between the tunica intima and a lumen of the carotid artery (herein referred to as a lumen-intima interface), and an interface between the tunica adventitia and the tunica media (herein referred to as a media-adventitia interface), as illustrated in FIG. 15B. A state in which IMT exceeds a specific value in a localized area is referred to as a plaque. A plaque leads to a vascular wall structure in a longitudinal cross-sectional image such as illustrated in FIG. 15D. Depending on size of the plaque, medicinal treatment or surgical treatment to remove the plaque may be required. Therefore, measurement of IMT or percentage stenosis of the blood vessel, which is affected by the plaque, is vital for diagnosis. In particular, blood flow velocity in a region in which a stenosis is present can be used as an indicator of percentage stenosis of a blood vessel. Blood flow velocity can be measured using Doppler ultrasound.

Measurement of blood flow velocity by Doppler ultrasound is valuable in diagnosing stenosis of a blood vessel. Unfortunately, in measurement by Doppler ultrasound a high degree of procedural skill is required in order to set width, position, and direction of a Doppler gate. In consideration of the above, a method is proposed which enables measurement by Doppler ultrasound, even without a high degree of procedural skill, by automating measurement.

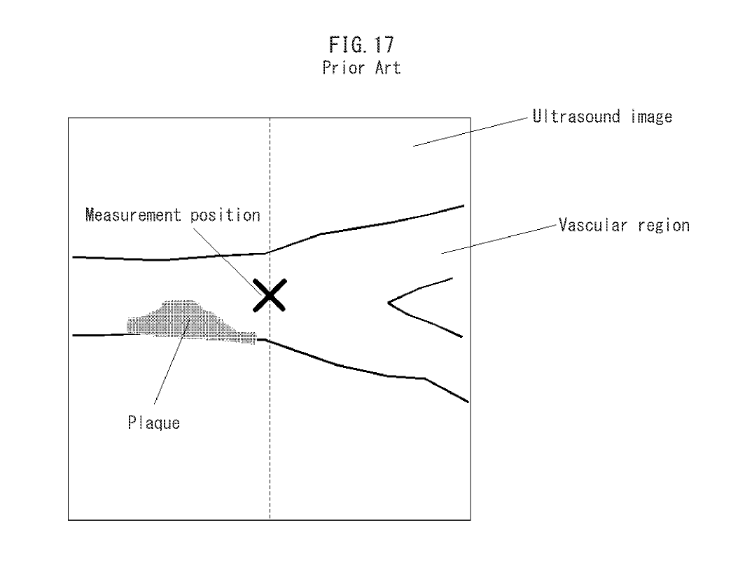

For example, in a conventional ultrasound diagnostic apparatus, a vascular region is detected from a B-mode ultrasound image and a measurement position for Doppler ultrasound is determined to be a predetermined position in the vascular region. FIG. 17 illustrates an example of determination of a measurement position by the conventional ultrasound diagnostic apparatus. In the example illustrated in FIG. 17, the measurement position is determined to be a center point of the vascular region. Consequently, the measurement position does not necessarily correspond to a position at which a plaque is present, and thus the measurement position which is determined cannot be used in order to measure blood flow velocity at a position corresponding to the plaque. Therefore, as described above, the conventional ultrasound diagnostic apparatus experiences a problem of being unable to measure blood flow velocity at a desired position.

In consideration of the above problem, the inventors conceived an idea of detecting a specific part of a blood vessel, such as a plaque or bifurcation structure, and subsequently performing measurement by Doppler ultrasound at a measurement position determined in accordance with the specific part which is detected. Through the above, a position of a diseased part, such as a plaque, can be automatically detected and blood flow velocity can be calculated by performing measurement by Doppler ultrasound at the detected position. Therefore, such a configuration enables simple and reproducible measurement of blood flow velocity, for example in a blood vessel.

### EMBODIMENTS

The following explains embodiments of the present invention with reference to the drawings.

### First Embodiment

<Configuration>

An ultrasound diagnostic apparatus, and a control method thereof, relating to a first embodiment are explained with reference to the drawings. An ultrasound diagnostic apparatus 10 relating to the first embodiment automatically determines position of a plaque or other part of a blood vessel based on shape of the blood vessel extracted from an ultrasound image, and performs measurement by Doppler ultrasound at the position which is determined. Herein, the term ultrasound image refers to a video image or a still image such as a B-mode ultrasound image, which is generated from a reception signal acquired by scanning a measurement target using an ultrasound probe. The term ultrasound image may also refer to a reception signal, or a signal resulting from processing of the reception signal, which is inclusive of sufficient information for generating a video image or a still image.

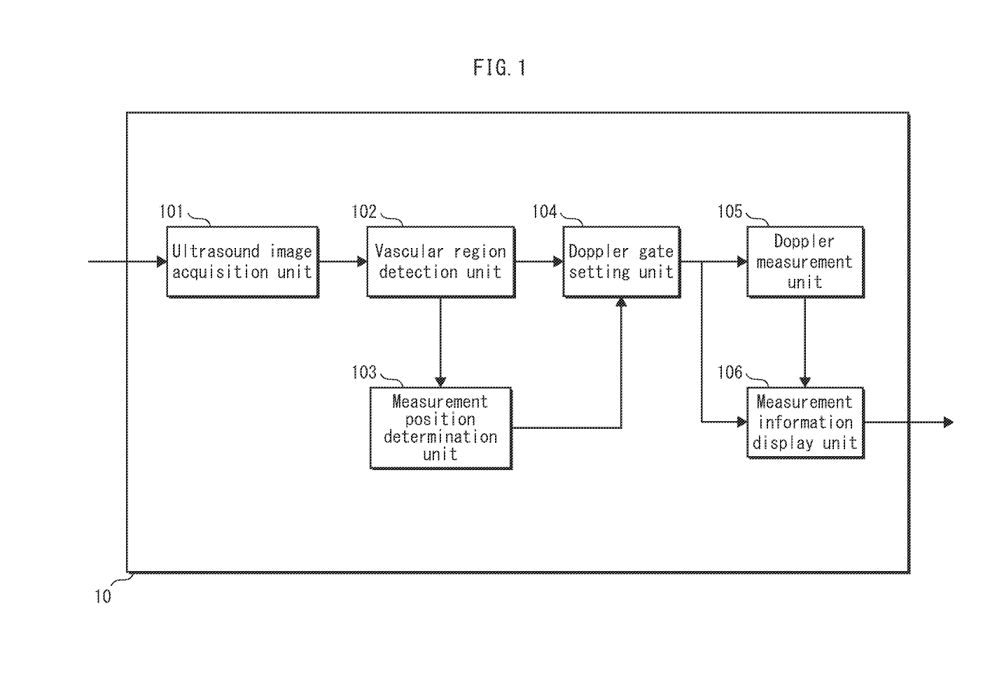

FIG. 1 is a block diagram illustrating configuration of the ultrasound diagnostic apparatus 10. The ultrasound diagnostic apparatus 10 includes an ultrasound image acquisition unit 101, a vascular region detection unit 102, a measurement position determination unit 103, a Doppler gate setting unit 104, a Doppler measurement unit 105, and a measurement information display unit 106.

The ultrasound image acquisition unit 101 generates a B-mode ultrasound image from a reception signal acquired by scanning a measurement target using an ultrasound probe, and outputs the B-mode ultrasound image to the vascular region detection unit 102. In the present embodiment, the ultrasound probe is a linear probe including a 1D array of ultrasound transducers that are for example made of lead zirconate titanate (PZT). Also, in the present embodiment the measurement target is a carotid artery.

The vascular region detection unit 102 detects a vascular wall of the carotid artery captured in the B-mode ultrasound image output by the ultrasound image acquisition unit 101, and extracts a vascular region. More specifically, the vascular region detection unit 102 detects a media-adventitia interface and a lumen-intima interface of the vascular wall. Herein, the term vascular region refers to a region inwards of a tunica adventitia of a blood vessel. In other words, the vascular region is composed of a vascular wall region and a lumen region. The vascular region detection unit 102 performs edge detection or the like on the B-mode ultrasound image based on changes in brightness values therein, and detects interfaces by for example taking into account continuity of edges which are detected.

The measurement position determination unit 103 detects a specific part of the vascular region based on the vascular region detected by the vascular region detection unit 102. The measurement position determination unit 103 determines one or more measurement positions for Doppler ultrasound in the vascular region, in terms of the longitudinal direction, in accordance with the specific part. In the present embodiment the specific part of the vascular region corresponds to a plaque in the measurement target. First, explanation is provided relating to detection of the specific part. The measurement position determination unit 103 calculates IMT using the lumen-intima interface and the media-adventitia interface detected by the vascular region detection unit 102, and detects a region in which IMT exceeds a predetermined value to correspond to the plaque. More specifically, the measurement position determination unit 103 calculates IMT, for each point along the media-adventitia interface, using a distance between the point on the media-adventitia interface and a corresponding point on the lumen-intima interface. Next, the measurement position determination unit 103 determines a measurement position for Doppler ultrasound in terms of the longitudinal direction of vascular region. The measurement position determination unit 103 determines a position of maximum hypertrophy to be the measurement position. The position of maximum hypertrophy is a position at which IMT is a maximum value in the region corresponding to the plaque.

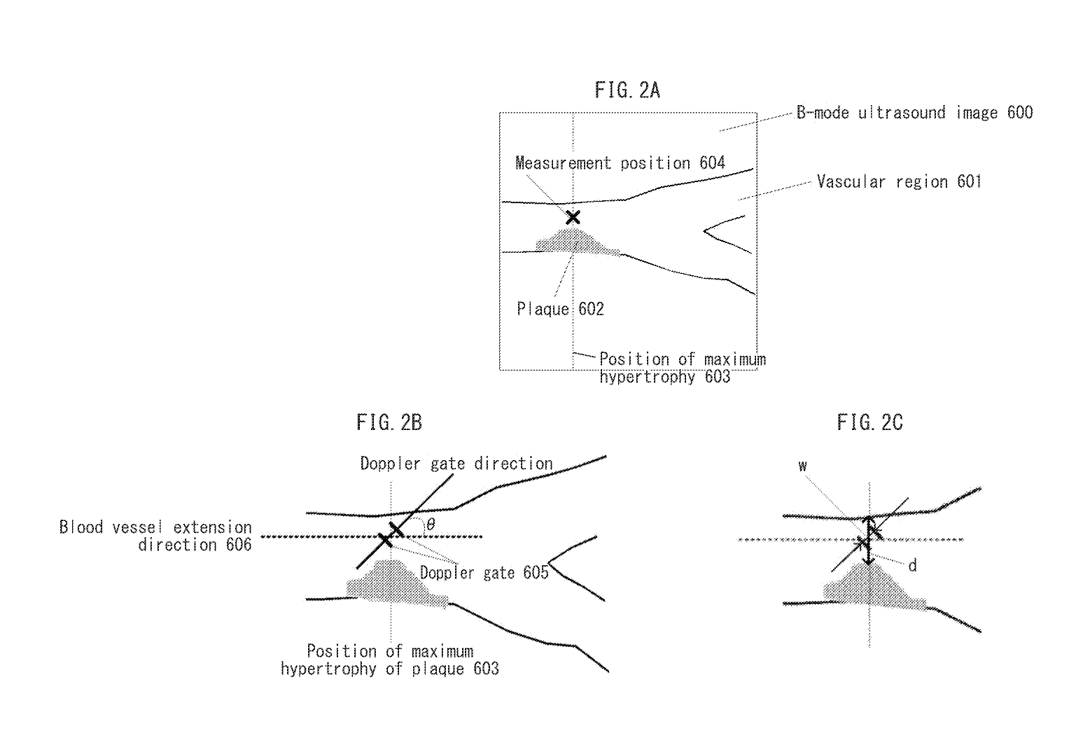

The following provides more specific explanation with reference to FIGS. 2A-2C. FIG. 2A illustrates an example in which a measurement position 604 is determined with respect to a B-mode ultrasound image 600, which is one example of an ultrasound image. The vascular region detection unit 102 detects a vascular region 601 from the B-mode ultrasound image 600. Next, the measurement position determination unit 103 detects a plaque 602 as a specific part based on the vascular region 601. Finally, the measurement position determination unit 103 identifies a position of maximum hypertrophy 603 and determines the measurement position 604 in correspondence with the position of maximum hypertrophy 603 which is identified.

The Doppler gate setting unit 104 sets a Doppler gate 605 in order to measure blood flow velocity at the measurement position. More specifically, the Doppler gate setting unit 104 sets the Doppler gate 605 such that at the measurement position 604 in the lumen, an angle θ between a direction of the Doppler gate 605 and a blood vessel extension direction relative to position of the Doppler gate 605 is no greater than a predetermined value. In the present embodiment the predetermined value is for example 60°. FIG. 2B illustrates an overview of setting of the Doppler gate. A center point of the Doppler gate 605 is set as a center point of an area surrounded by the lumen-intima interface at the position of maximum hypertrophy 603. Direction of the Doppler gate 605 corresponds to a direction in which ultrasound travels when transmitted towards the Doppler gate 605 from the ultrasound probe during Doppler ultrasound measurement. Ultrasound can be emitted along a plane having a specific orientation by creating a phase difference using a delay time between transmission of ultrasound from neighboring ultrasound transducers. Based on a blood vessel extension direction 606 detected by the vascular region detection unit 102 and the measurement position 604, the Doppler gate setting unit 104 judges whether the angle θ between the direction of the Doppler gate 605 and the blood vessel extension direction 606 is no greater than 60°. Direction of the Doppler gate 605 corresponds to a direction between a current position of the ultrasound probe and the measurement position 604. When the angle θ is no greater than 60°, the Doppler gate setting unit 104 sets direction of the Doppler gate 605 as the direction between the current position of the ultrasound probe and the measurement position 604. Note that when the angle θ is greater than 60°, the ultrasound diagnostic apparatus 10 displays guidance that urges a user to change position or orientation of the ultrasound probe.

Next, width w of the Doppler gate 605 is explained with reference to FIG. 2C. Width w of the Doppler gate 605 is set using diameter d of the lumen at the position of maximum hypertrophy 603 as a reference, such that, for example, a relationship w=d/2 is satisfied.

The Doppler measurement unit 105 measures blood flow velocity in accordance with the Doppler gate 605 set by the Doppler gate setting unit 104.

The measurement information display unit 106 is for example configured by a liquid-crystal display. The measurement information display unit 106 displays the B-mode ultrasound image generated by the ultrasound image acquisition unit 101 and with the Doppler gate 605 set by the Doppler gate setting unit 105 superposed thereon. The measurement information display unit 106 also displays the blood flow velocity measured by the Doppler measurement unit 105.

<Operation>

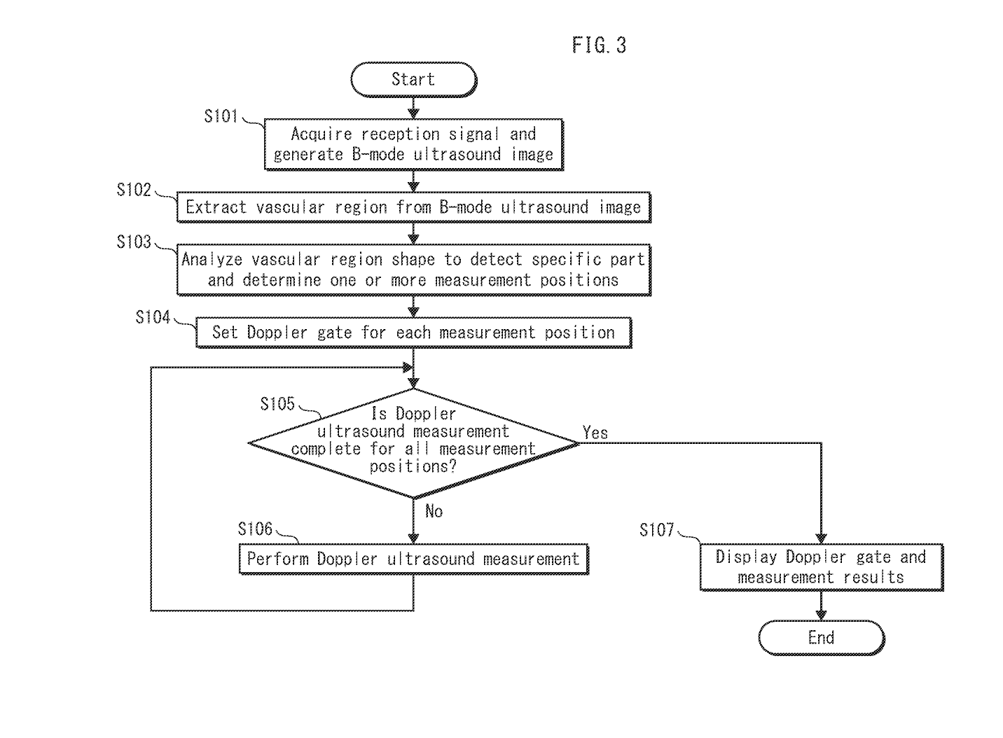

FIG. 3 is a flowchart illustrating operation of the ultrasound diagnostic apparatus 10.

The ultrasound image acquisition unit 101 first acquires a reception signal and generates a B-mode ultrasound image (Step S101).

Next, the vascular region detection unit 102 extracts a vascular region from the B-mode ultrasound image generated by the ultrasound image acquisition unit 101 (Step S102). During the above, the vascular region detection unit 102 detects both shape of a vascular wall region in the vascular region and also a blood vessel extension direction.

The measurement position determination unit 103 subsequently detects a specific part of the vascular region based on output from the vascular region detection unit 102, and determines one or more measurement positions (Step S103).

Next, the Doppler gate setting unit 104 sets a Doppler gate with respect to each of the measurement positions that is determined by the measurement position determination unit 103 (Step S104).

The ultrasound diagnostic apparatus 10 subsequently judges whether Doppler ultrasound measurement is complete for all of the Doppler gates set by the Doppler gate setting unit 104 (Step S105). When the ultrasound diagnostic apparatus 10 judges that a Doppler gate has been set for which measurement is not complete, the Doppler measurement unit 105 performs Doppler ultrasound measurement in order to measure blood flow velocity in the carotid artery in accordance with the Doppler gate which is set (Step S106), and subsequently the ultrasound diagnostic apparatus 10 repeats performance of Step S105.

On the other hand, when Doppler ultrasound measurement is complete for all of the measurement positions, the measurement information display unit 106 displays the Doppler gates and measurement results (Step S107). More specifically, measurement information display unit 106 displays the Doppler gates and the measurement results for blood flow velocity at the Doppler gates, superposed on the B-mode ultrasound image generated by the ultrasound image acquisition unit 101.

<Conclusion>

As described above, the ultrasound diagnostic apparatus 10 relating to the present embodiment analyzes shape of a vascular wall of a blood vessel and automatically sets an optimum measurement position for Doppler ultrasound with respect to a disease affected part of the blood vessel, such as a plaque. Therefore, the ultrasound diagnostic apparatus 10 enables simple and highly reproducible measurement by Doppler ultrasound.

<Supplementary Explanation>

(1) In the first embodiment, the measurement target is a carotid artery, but the measurement target is not limited to being the carotid artery. Alternatively, measurement of blood flow velocity during diagnosis may be performed on a different blood vessel such as an abdominal aorta or a tibial artery, or on a different body part such as the heart.

(2) In the first embodiment, the ultrasound probe is a linear probe, but the ultrasound probe may alternatively be a convex probe or a sector probe. Further alternatively, the ultrasound probe may be a probe that can acquire an ultrasound image of a 3D region beneath the probe, such as an oscillating probe in which the ultrasound transducers oscillate, or a probe in which ultrasound transducers are arranged in a matrix as a 2D array.

(3) In the first embodiment, the ultrasound image acquisition unit 101 generates a B-mode ultrasound image and the vascular region detection unit 102 detects a vascular wall region from the B-mode ultrasound image, but the present invention is not limited to such a configuration. For example, alternatively the ultrasound image acquisition unit 101 may output a reception signal acquired from the ultrasound probe, without generating a B-mode ultrasound image, and the measurement position determination unit 103 may detect a vascular wall region from the reception signal. Further alternatively, the ultrasound image acquisition unit 101 may acquire an ultrasound image that indicates blood flow information such as a color flow image or a power mode image. In such a configuration, the vascular region detection unit 102 detects a vascular region by determining that a region in which blood flow velocity is at least a predetermined value corresponds to a lumen of a blood vessel. The following explains reasoning as to why a Doppler gate is set even though blood flow velocity can be acquired from a color flow image or a power mode image. Color flow and power mode images are used mainly in order to observe whether or not blood flow occurs in a certain region and to measure a rough magnitude of blood flow velocity. The blood flow velocity is measured as an average value for blood flow velocity in the region using a basic method such as autocorrelation of an ultrasound signal. Such a method has a comparatively low level of accuracy compared to measurement by Doppler ultrasound which uses FFT (Fast Fourier Transform) or the like to accurately measure blood flow velocity in a localized region.

Note that alternatively both a B-mode ultrasound image and an image indicating blood flow may be used in combination. For example, when a boundary of tissue such as muscle is present, the boundary is detected from a B-mode ultrasound image as a smooth edge where a large difference in brightness values occurs, and thus differentiation between the boundary of the tissue and a vascular wall is difficult using the B-mode ultrasound image. In such a situation it is possible to differentiate between whether a certain edge is a vascular wall or a boundary of a different tissue by investigating whether blood flows in a region inside of the edge. Furthermore, due to the fact that a blood flow image can only be used to detect a lumen-intima interface, the lumen-intima interface detected from the blood flow image may be used in combination with a media-adventitia interface detected from a B-mode ultrasound image. Note that although detection of the lumen-intima interface and the media-adventitia interface is performed automatically, the user may manually correct position of an interface when an error occurs during the automatic detection. Manual correction is particularly effective in regards to the lumen-intima interface, which is often extracted unclearly.

(4) In the first embodiment, the vascular region detection unit 102 detects a vascular region from a B-mode ultrasound image and outputs information relating to the vascular region, but the vascular region detection unit 102 may also perform verification on the B-mode ultrasound image. For example, when a vascular region is not detected from a B-mode ultrasound image or when a vascular wall region in the B-mode ultrasound image is not continuous, the B-mode ultrasound image may be considered to be unclear and ultrasound image acquisition in Step S101 may be repeated. A configuration such as described above prevents measurement position determination being performed based on a B-mode ultrasound image which is acquired while the ultrasound probe is being moved in order to find a scan position at which a longitudinal cross-sectional image of the blood vessel can be acquired, and consequently from which rendering of an image of the blood vessel is incomplete or unclear.

Furthermore, ultrasound image acquisition in Step S101 may also be repeated when the B-mode ultrasound image is not a longitudinal cross-sectional image of the blood vessel. Judgment can be performed as to whether an acquired ultrasound image is a longitudinal cross-sectional image, based for example on the fact that in a longitudinal cross-sectional image, the vascular region is a continuous region of at least a certain length. The above ensures that a longitudinal cross-sectional image of the blood vessel is used during measurement by Doppler ultrasound.

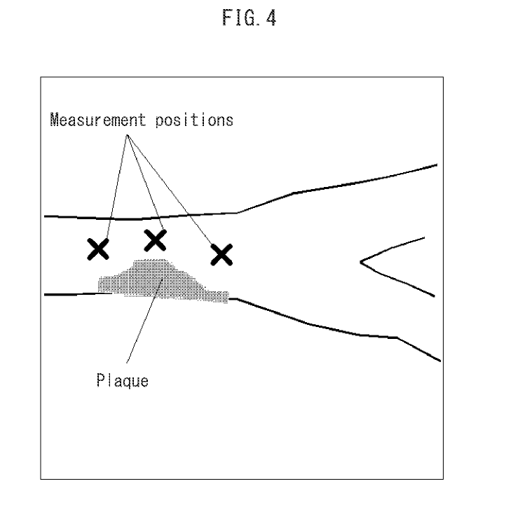

(5) In the first embodiment, a single measurement position for Doppler ultrasound is determined in accordance with a plaque, but the present invention is not limited to such a configuration. Alternatively, a plurality of measurement positions for Doppler ultrasound may be determined in accordance with a single plaque. FIG. 4 illustrates an example in which a plurality of measurement positions are determined for Doppler ultrasound. In the present example three measurement positions are determined for Doppler ultrasound; a start position of the plaque, a position of maximum hypertrophy, and an end position of the plaque. Performing measurement at a plurality of measurement positions enables acquisition of more accurate information, such as maximum blood flow velocity in a region corresponding to the plaque. Note that the number of measurement positions for Doppler ultrasound which are determined in accordance with the plaque is not limited to one or three, and alternatively a greater number of measurement positions may be determined. In proximity to the start point or the end point of the plaque, blood flow is irregular and turbulence tends to occur. A distribution of blood flow velocity is irregular in a region in which turbulence occurs. Turbulence can be detected based on measurement results of blood flow velocity measured at a plurality of measurement positions. Consequently, it is possible to judge whether a plaque is present based on whether or not turbulence is detected.

(6) In the first embodiment, a center point of a Doppler gate is determined to be a center point of an area surrounded by a lumen-intima interface, but alternatively the center point of the Doppler gate may be determined to be a center point of an area surrounded by a media-adventitia interface.

Also, in the first embodiment, width w of the Doppler gate is equivalent to half of diameter d of the lumen at the position of maximum hypertrophy, but alternatively the width w of the Doppler gate may, for example, satisfy a relationship w=d/(2 cos θ).

(7) In the first embodiment, the ultrasound diagnostic apparatus 10 displays guidance urging the user to change position or orientation of the ultrasound probe when the angle θ between the Doppler gate direction (in other words, the direction between the current position of the ultrasound probe and the measurement position) and the blood vessel extension direction exceeds 60°. Alternatively, the measurement information display unit 106 may display information that indicates an amount that the current position or the current orientation of the ultrasound probe should be changed in order that the angle θ does not exceed a predetermined value.

(8) In the first embodiment, Steps S105 and S106 are repeated when measurement by Doppler ultrasound is to be performed at a plurality of measurement positions, but alternatively processing may be performed as described below. In the alternative processing, when the measurement position determination unit 103 determines a plurality of measurement positions in Step S103, the measurement position determination unit 103 stores the measurement positions and outputs one of the plurality of measurement positions to the Doppler gate setting unit 104. Once measurement of blood flow velocity for a Doppler gate corresponding to the one measurement position is completed in Step S106, Step S101 is repeated, and in Step S103 the measurement position determination unit 103 outputs a next one of the plurality of measurement positions, at which blood flow velocity has not yet been measured. Through the processing described above, when the B-mode ultrasound image changes between successive measurements, a Doppler gate can be set in the lumen in accordance with the aforementioned change. Reasoning behind the above is that measurement at a single measurement position by Doppler ultrasound requires an amount of time equivalent to several heat beats, and consequently position of the ultrasound probe may change or the subject may move between measurements.

(9) In the first embodiment only a single B-mode ultrasound image is generated, but alternatively ultrasound image acquisition or blood flow velocity measurement may be performed a plurality of times in order to acquire more reliable results.

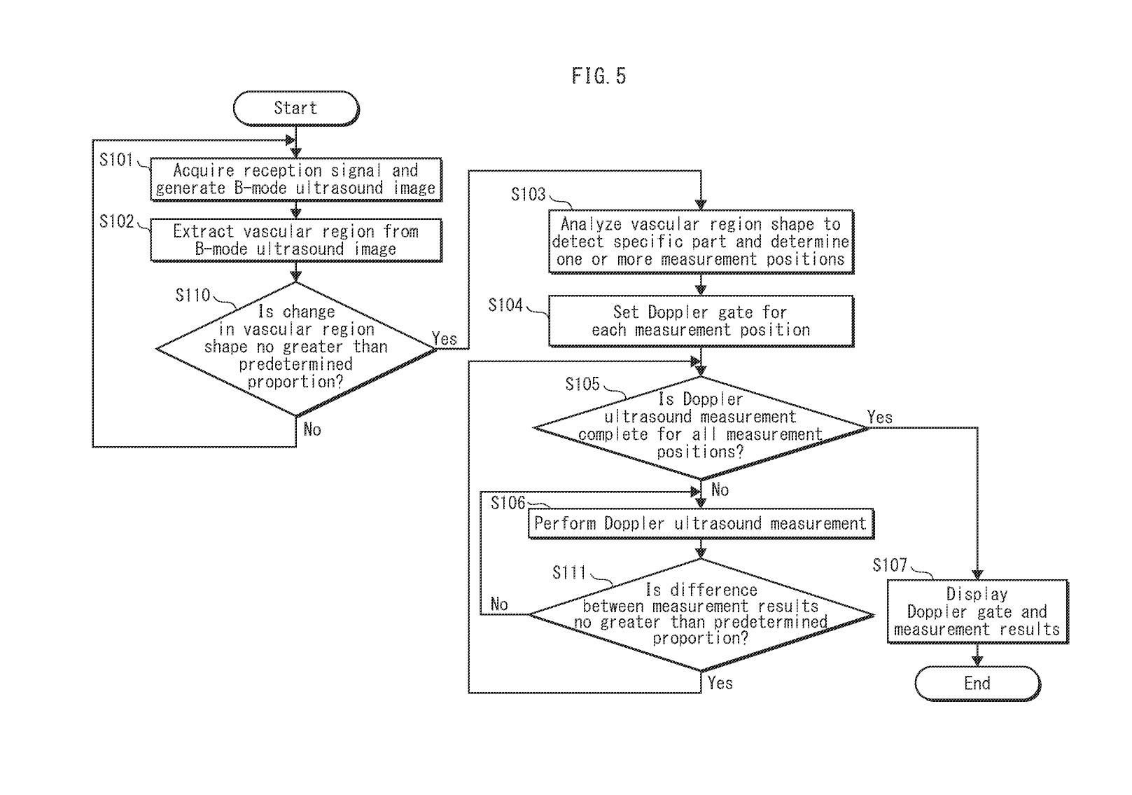

The following explanation refers to the flowchart illustrated in FIG. 5. Steps in FIG. 5 which are the same as steps in FIG. 3 are indicated using the same reference signs and explanation thereof is omitted.

Once extraction of a vascular region by the vascular region detection unit 102 has been completed in Step S102, the ultrasound diagnostic apparatus 10 repeats Step S101 in order to acquire an additional B-mode ultrasound image. The ultrasound diagnostic apparatus 10 compares shapes of respective vascular regions extracted from the two ultrasound images which have been acquired (Step S110). When change in vascular region shape between the two ultrasound images exceeds a predetermined proportion, the ultrasound diagnostic apparatus 10 repeats Step S101 in order to acquire an additional ultrasound image. When change in vascular region shape between the two ultrasound images does not exceed the predetermined proportion, operation of the ultrasound diagnostic apparatus 10 proceeds to Step S103. The above configuration enables judgment as to whether the user has completed searching for a blood vessel image. Reasoning for the above is that when the user is searching, while moving the ultrasound probe, in order that a desired blood vessel image is rendered, scan position of the ultrasound probe changes, causing a large amount of change in vascular region shape between successive ultrasound images. On the other hand, once searching for a desired blood vessel image is completed, the ultrasound probe is held stationary such that only a small amount of change in vascular region shape occurs between successive ultrasound images.

Once Doppler ultrasound measurement has been performed in Step S106, the ultrasound diagnostic apparatus 10 repeats Step S106 in order to perform an additional measurement for the same Doppler gate, and compares results of the two measurements (Step S111). When a difference between the two measurements exceeds a predetermined proportion, the ultrasound diagnostic apparatus 10 repeats Step S106. When the difference does not exceed the predetermined proportion, measurement is complete for the aforementioned Doppler gate, and operation of the ultrasound diagnosis apparatus 10 returns to Step S105. The configuration described above improves reliability of results obtained during measurement by Doppler ultrasound.

Note that alternatively only one out of Steps S110 and S111 may be performed.

Further alternatively, three or more ultrasound images that have been acquired may be compared in Step S110, and results of three or more measurements by Doppler ultrasound may be compared in Step S111. Note that in Step S111 all results obtained during measurement by Doppler ultrasound may be output, or alternatively only a last result may be output. Further alternatively, a representative value may be output, such as a mean value or a median value of the plurality of measurements by Doppler ultrasound. In a configuration in which a representative value is output, when a difference between results of a certain measurement and other measurements exceeds a predetermined proportion, the result of the aforementioned measurement may be treated as an outlier and be excluded from use.

### Modified Example

In the first embodiment, a plaque is detected from the vascular region as the specific part and one or more measurement positions are determined in proximity to the plaque. In the present modified example, explanation is given of a configuration in which blood flow velocity is measured by determining one or more measurement positions based on typical shape of a part of a vascular wall which is susceptible to formation of a plaque.

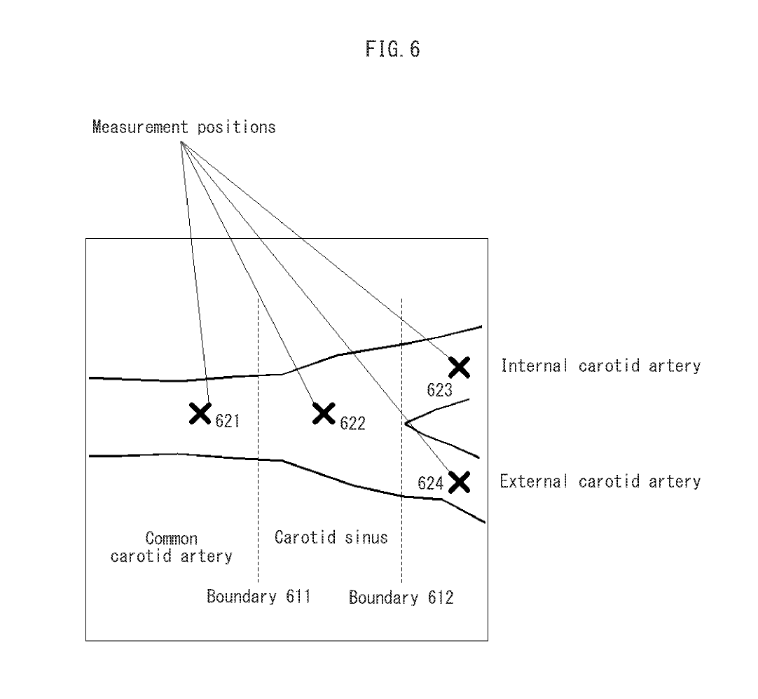

FIG. 6 is a longitudinal cross-sectional image of the carotid artery that illustrates an example of determining measurement positions. The measurement position determination unit 103 detects, as specific parts, a boundary 611 between the common carotid artery and the carotid sinus, and a boundary 612 between the carotid sinus and the internal and external carotid arteries, based on shape of the media-adventitia interface. The boundary 611 between the common carotid artery and the carotid sinus is detected based on change, in terms of the longitudinal direction, in values for blood vessel diameter or blood vessel cross-sectional area, or based on derivatives of such values (i.e., rates of change). The above is due to blood vessel diameter increasing in the carotid sinus, from the boundary 611 between the common carotid artery and the carotid sinus, to a start point of the internal carotid artery or the external carotid artery, whereas blood vessel diameter in the common carotid artery is approximately constant. The boundary 612 between the carotid sinus and the internal and external carotid arteries is for example detected based on cross-sectional shape of the blood vessel or on a number of blood vessel cross-sections which are present. The above is due to the boundary 612 corresponding to a bifurcation at which a number of blood vessels increases from one to two.

Next, the measurement position determination unit 103 determines measurement positions based on the boundaries 611 and 612. More specifically, the measurement position determination unit 103 determines a measurement position 621 in the common carotid artery that is predetermined distance from the boundary 611. The measurement position determination unit 103 also determines a measurement position 622 in the carotid sinus that is midway between the boundaries 611 and 612. The measurement position determination unit 103 also determines measurement positions 623 and 624 in the internal and external carotid arteries respectively, that are each a predetermined distance from the boundary 612.

Through measurement of blood flow velocity at a representative position in each of the parts of the carotid artery as described above, it is possible to judge whether a plaque is present in the part. Note that determination of measurement positions can be adjusted in accordance with a diagnostic objective, for example by determining a plurality of measurement positions in a part of the carotid artery which is susceptible to formation of a plaque, such as the carotid sinus. Furthermore, the common carotid artery, the carotid sinus and the internal carotid artery are most important in terms of diagnosis, and thus alternatively measurement positions may only be determined in the aforementioned parts. In a situation in which only the common carotid artery and the carotid sinus are rendered in the B-mode ultrasound image, measurement positions may only be determined in the aforementioned rendered parts, or warning information may be displayed indicating that the internal carotid artery has not been rendered.

(Supplementary Explanation)

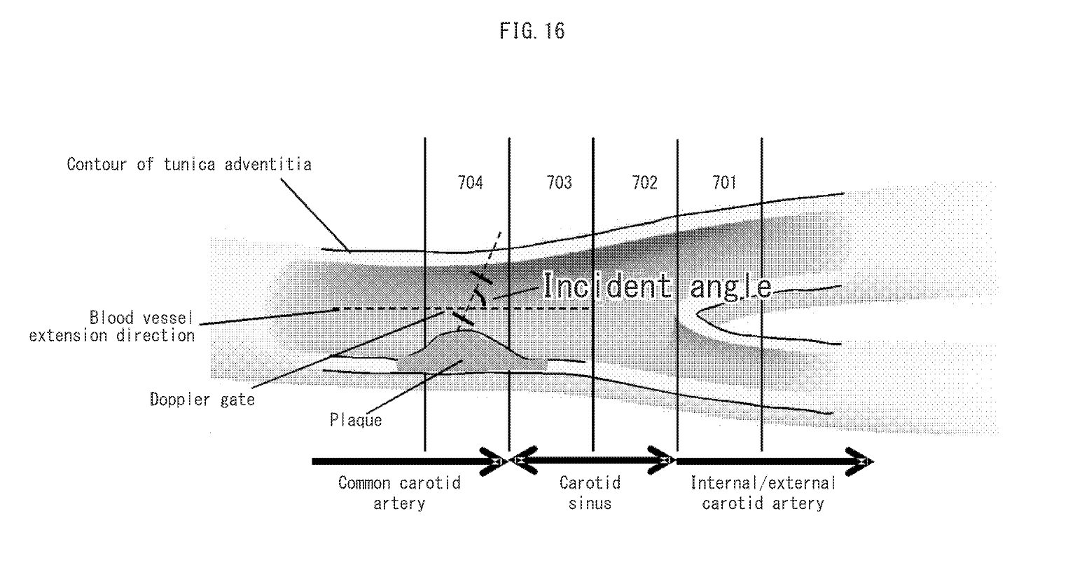

(1) In the modified example of the first embodiment, the boundary 611 between the common carotid artery and the carotid sinus, and the boundary 612 between the carotid sinus and the internal and external carotid arteries, are both used when performing segmentation of the carotid artery. Alternatively, as illustrated in FIG. 16, a bifurcation may be detected as a specific part of the carotid artery, and the bifurcation may be used as a reference for defining four segments 701-704, each having a length of 1 cm.

### Second Embodiment

An ultrasound diagnostic apparatus 20 relating to a second embodiment differs from the ultrasound diagnostic apparatus 10 in terms that the ultrasound diagnostic apparatus 20 determines a measurement position for Doppler ultrasound using a pre-acquired 3D image of a body part which is a measurement target. Note that elements configuring the ultrasound diagnostic apparatus 20 which are the same as in the first embodiment are labeled using the same reference signs and explanation thereof is omitted.

<Configuration>

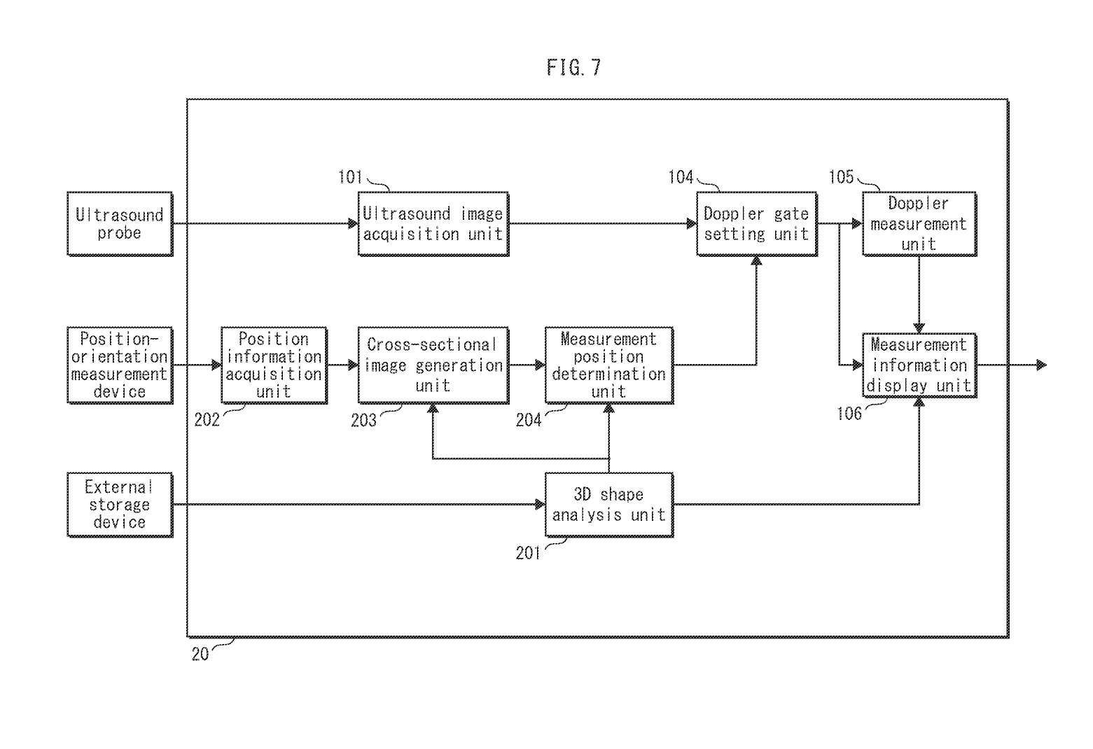

FIG. 7 is a block diagram illustrating configuration of the ultrasound diagnostic apparatus 20. The ultrasound diagnostic apparatus 20 includes an ultrasound image acquisition unit 101, a Doppler gate setting unit 104, a Doppler measurement unit 105, a measurement information display unit 106, a 3D shape analysis unit 201, a position information acquisition unit 202, a cross-sectional image generation unit 203, and a measurement position determination unit 204.

The following explains examination of the carotid artery using the ultrasound diagnostic apparatus 20, focusing on operation of the 3D shape analysis unit 201, the position information acquisition unit 202, the cross-sectional image generation unit 203, and the measurement position determination unit 204, which are features of the ultrasound diagnostic apparatus 20.

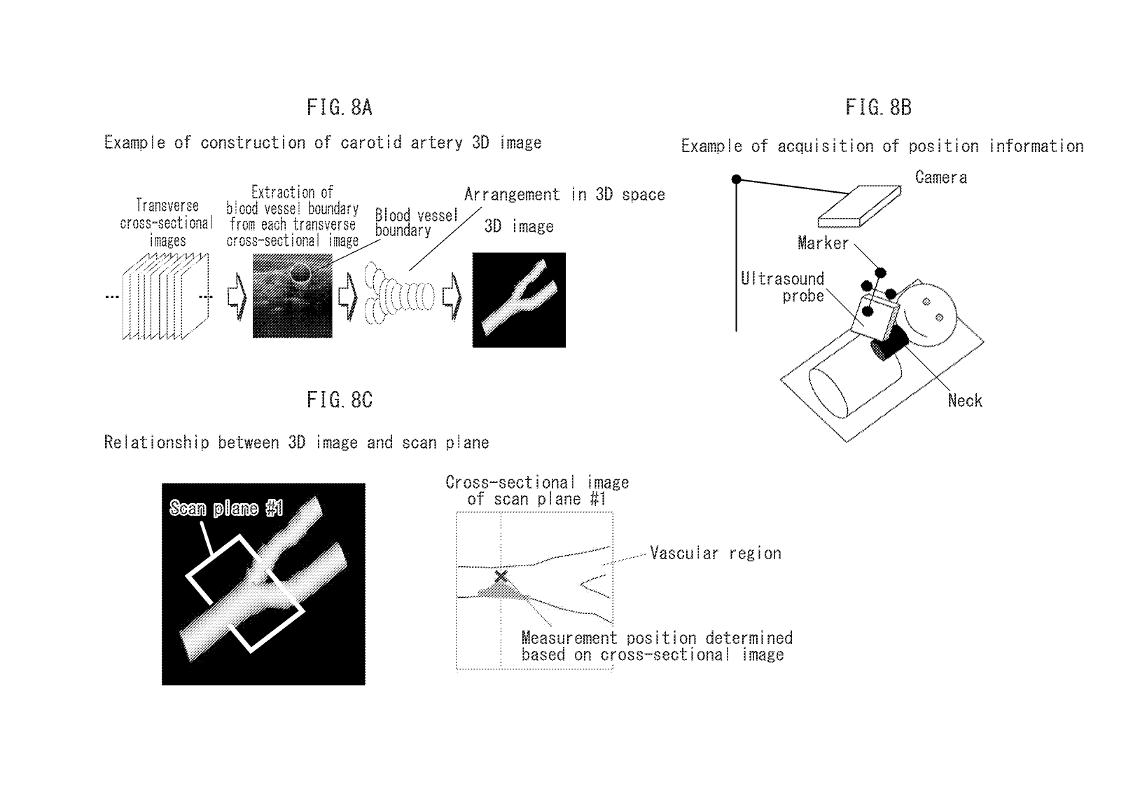

The 3D shape analysis unit 201 acquires, from an external storage device, a 3D image illustrating shape of a lumen-intima interface or a media-adventitia interface of a carotid artery and detects a plaque by analyzing the 3D image. The 3D image may for example be constructed from successively acquired B-mode ultrasound images, MRI, or x-ray computed tomography (CT). FIG. 8A illustrates an example in which a 3D image of the carotid artery is constructed using a plurality of B-mode ultrasound images that are each a transverse cross-sectional image of the carotid artery. First a scan is performed along an entire length of the carotid artery in the longitudinal direction, successively acquiring a plurality of transverse cross-sectional images while also acquiring position information. Next, the lumen-intima interface or the media-adventitia interface is extracted from each of the transverse cross-sectional images. Finally, a 3D image is acquired by arranging the lumen-intima interfaces or the media-adventitia interfaces that have been extracted in a 3D space in accordance with the position information corresponding to each of the transverse cross-sectional images. The 3D image is stored in the external storage device.

The position information acquisition unit 202 measures position and orientation of the ultrasound probe. More specifically, position and orientation of the ultrasound probe are measured by using a camera to capture an image of three or more optical markers that are attached to the ultrasound probe in advance, and by measuring position and orientation based on size of the optical markers and relative positions of the optical markers in the image captured by the camera. The optical markers may for example each be a light-emitter such as an LED, or a reflector, but note that there is no particular limitation on configuration of the optical markers, so long as position of the optical markers can be captured by the camera. FIG. 8B illustrates an example in which four optical markers, indicated by black dots, are attached to the ultrasound probe and the position information is acquired by a camera capturing an image of the optical markers.

Coordinates acquired by the position information acquisition unit 202, indicating position and orientation of the ultrasound probe, and coordinates of the 3D image analyzed by the 3D shape analysis unit 201 are calibrated such as to be in correspondence. For example, in a situation in which, as described above, the 3D image is constructed from B-mode ultrasound images that are transverse cross-sectional images of a blood vessel, for each of the B-mode ultrasound images, the position information acquisition unit 202 acquires position and orientation of the ultrasound probe at a time at which the corresponding B-mode ultrasound image was captured, and stores the position and orientation with the B-mode ultrasound image.

The cross-sectional image generation unit 203 detects a plane from the 3D image that passes centrally through the blood vessel in the 3D image, and generates a longitudinal cross-sectional image that is a cross-sectional image along the plane. When the longitudinal cross-sectional image is generated, the ultrasound probe is guided such that a scan plane for a current position and a current orientation of the ultrasound probe matches the longitudinal cross-sectional image. More specifically, the measurement information display unit 106 is used in order to display information indicating an amount that position or orientation of the ultrasound probe should be changed from the current position and the current orientation in order that the scan plane of the ultrasound probe matches the plane of the longitudinal cross-sectional image. FIG. 8C illustrates an example of a longitudinal cross-sectional image of the 3D image. Scan plane #1 indicates the scan plane of the ultrasound probe after guidance thereof.

The measurement position determination unit 204 determines a measurement position for Doppler ultrasound in a current scan plane of the ultrasound probe, in accordance with a specific part such as a plaque detected by the 3D shape analysis unit 201.

<Operation>

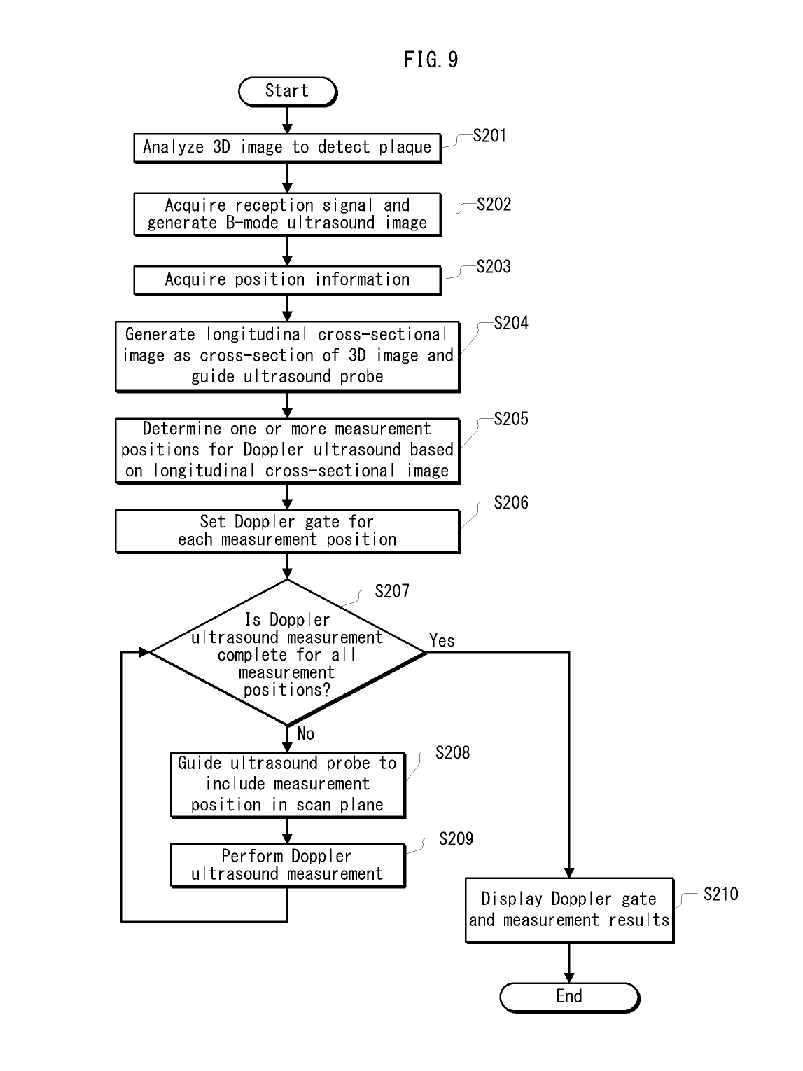

FIG. 9 is a flowchart illustrating operation of the ultrasound diagnostic apparatus 20.

The 3D shape analysis unit 201 first acquires a 3D image from the external storage device, analyzes the 3D image, and detects a plaque as a specific part of a blood vessel in the 3D image (Step S201).

Next, the ultrasound image acquisition unit 101 acquires a reception signal and generates a B-mode ultrasound image (Step S202).

The position information acquisition unit 202 acquires a position and an orientation of the ultrasound probe corresponding to the B-mode ultrasound image generated in Step S202 (Step S203).

Next, the cross-sectional image generation unit 203 acquires the 3D image from the 3D shape analysis unit 201, and acquires the position and the orientation of the ultrasound probe from the position information acquisition unit 202. The cross-sectional image generation unit 203 detects a plane passing centrally through the blood vessel in the 3D image, generates a cross-sectional image which is a longitudinal cross-sectional image of the blood vessel, and guides the ultrasound probe such that a scan plane thereof matches the longitudinal cross-sectional image (Step S204).

The measurement position determination unit 204 subsequently determines one or more measurement positions in accordance with the longitudinal cross-sectional image generated in Step S204 and the specific part detected by the 3D shape analysis unit 201 (Step S205).

Next, the Doppler gate setting unit 104 sets a Doppler gate with respect to each of the measurement positions (Step S206). Detailed explanation is omitted due to Step S206 being the same as Step S104.

In order that measurement by Doppler ultrasound is performed for all of the Doppler gates, the ultrasound diagnostic apparatus 20 repeats guidance of the ultrasound probe (Step S208) and measurement by Doppler ultrasound (Step S209) until judging that measurement by Doppler ultrasound has been performed for all of the Doppler gates (Step S207). When at a current position and a current orientation of the ultrasound probe, a scan plane of the ultrasound probe does not match a cross-sectional image including the measurement position, a guidance operation (Step S208) is performed in order to guide the ultrasound probe to a position and an orientation at which the scan plane includes the measurement position. More specifically, the measurement information display unit 106 displays information indicating an amount that position or orientation of the ultrasound probe should be changed from the current position and the current orientation in order that the scan plane of the ultrasound probe matches the cross-sectional image. Once the ultrasound probe has been moved to an appropriate position, measurement by Doppler ultrasound is performed at a position corresponding to a Doppler gate which has been set (Step S209).

Once measurement by Doppler ultrasound has been completed for all of the measurement positions, the measurement information display unit 106 displays the Doppler gates and measurement results (Step S210). More specifically, the measurement information display unit 106 displays the Doppler gates and measurement results for blood flow velocity at each of the Doppler gates, superposed on the B-mode ultrasound image generated by the ultrasound image acquisition unit 101.

<Conclusion>

Through the above configuration, measurement by Doppler ultrasound can be performed based on a pre-acquired 3D image. Such a configuration enables determination of measurement positions as positions that are more suitable for measurement.

<Supplementary Explanation>

(1) In the second embodiment, a plaque is detected as a specific part of the blood vessel and a measurement position is set in proximity to the specific part. Alternatively, in the same way as in the modified example of the first embodiment, the 3D shape analysis unit 201 may detect a change in blood vessel diameter, a bifurcation of the blood vessel, or the like as the specific part, and the measurement position determination unit 204 may segment the blood vessel in accordance with the specific part and determine a measurement position with respect to each of the segments resulting from segmentation.

(2) In the second embodiment, the position information acquisition unit 202 uses a camera to capture an image of three or more optical markers attached to the ultrasound probe and measures size and relative positions of the optical markers in the image, but the above is not a limitation. For example, alternatively positions of the optical markers may be acquired using a stereo-camera, or may be acquired by a sensor other than a camera, such as a magnetic sensor, an acceleration sensor, or a gyroscope.

(3) In the second embodiment, the cross-sectional image generation unit 203 generates a cross-sectional image of the 3D image along a plane passing centrally through the blood vessel in the 3D image. Alternatively, the cross-sectional image generation unit 203 may generate a cross-sectional image corresponding to a scan plane of the ultrasound probe and perform verification on the cross-sectional image that is generated. For example, in a situation in which the scan plane is spatially separated from the 3D image such that the scan plane does not coincide with the 3D image, the measurement information display unit 106 may be used in order to display guidance that urges the user to change position or orientation of the ultrasound probe. The measurement information display unit 106 may alternatively display information indicating an amount that position or orientation of the ultrasound probe should be changed from a current position and a current orientation thereof in order that the scan plane of the ultrasound probe coincides with the 3D image. Furthermore, the measurement information display unit 106 may also urge the user to change position or orientation of the ultrasound probe in the same way in a situation in which the cross-sectional image which is generated is not a longitudinal cross-sectional image of the blood vessel.

(4) In the second embodiment, the measurement information display unit 106 displays the Doppler gates and measurement results superposed on the B-mode ultrasound image generated by the ultrasound image acquisition unit 101. Alternatively, the measurement information display unit 106 may also display superposed information indicating positional relationship to the 3D image as illustrated in FIG. 8C. Further alternatively, the measurement information display unit 106 may display the Doppler gates and measurement results superposed on the 3D image.

### Third Embodiment

An ultrasound diagnostic apparatus 30 relating to a third embodiment measures blood flow velocity at a representative position in each segment of a body part which is a measurement target, judges whether or not the blood flow velocity is normal, and thus detects a diseased part such as a plaque. The ultrasound diagnostic apparatus 30 differs from the ultrasound diagnostic apparatus 10 in terms that the ultrasound diagnostic apparatus 30 detects a diseased part based on blood flow velocity, whereas the ultrasound diagnostic apparatus 10 detects a diseased part based on shape of a lumen-intima interface or a media-adventitia interface in a B-mode ultrasound image, and subsequently measures blood flow velocity. Elements of configuration that are the same as in the first embodiment are labeled below using the same reference signs and explanation thereof is omitted.

<Configuration>

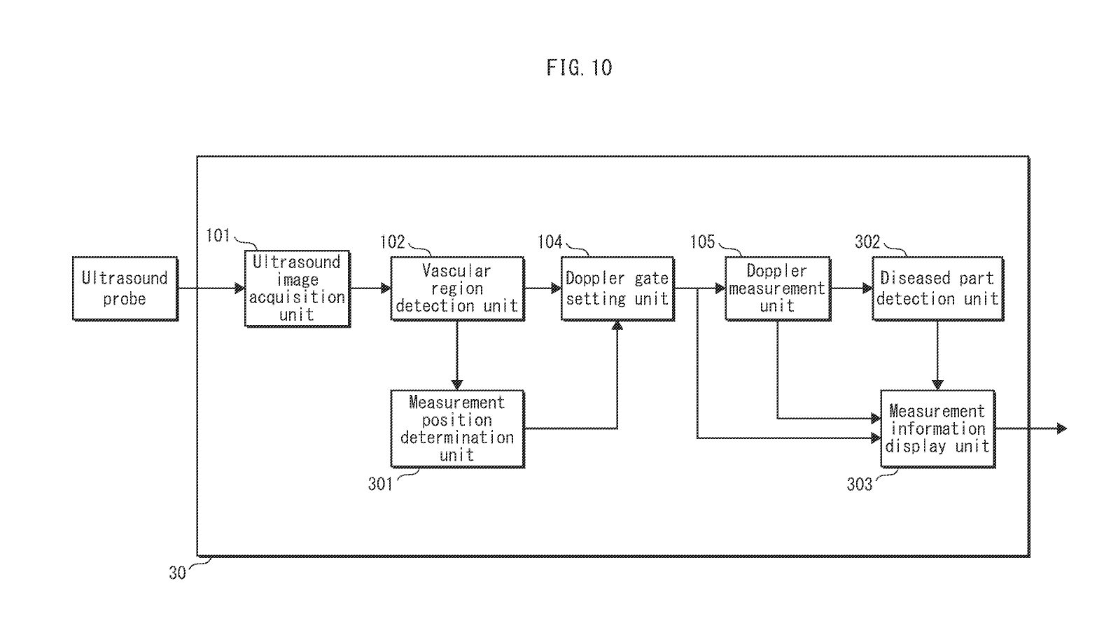

FIG. 10 is a block diagram illustrating configuration of the ultrasound diagnostic apparatus 30. The ultrasound diagnostic apparatus 30 includes an ultrasound image acquisition unit 101, a vascular region detection unit 102, a Doppler gate setting unit 104, a Doppler measurement unit 105, a measurement position determination unit 301, a diseased part detection unit 302, and a measurement information display unit 303. The following explains an example of examination of a carotid artery, focusing on operation of the measurement position determination unit 301, the diseased part detection unit 302, and the measurement information display unit 303 which are features of the ultrasound diagnostic apparatus 30.

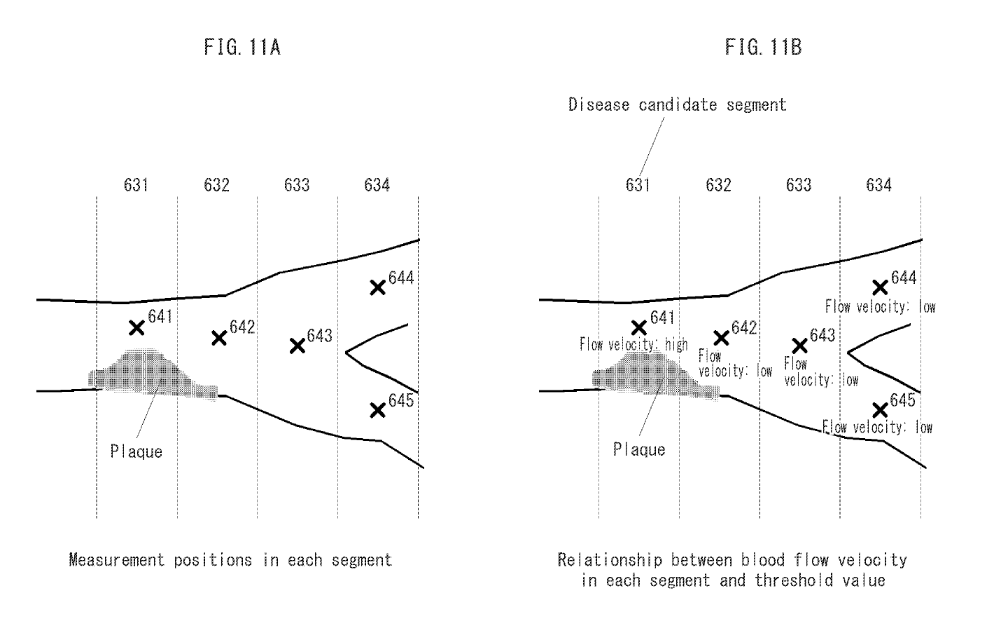

The measurement position determination unit 301 detects the boundary between the common carotid artery and the carotid sinus, or the bifurcation of the carotid artery (i.e., the boundary between the carotid sinus and the internal and external carotid arteries) as a specific part, performs segmentation of the carotid artery in accordance with the specific part, and determines a representative position in each of the segments to be a measurement position. As explained in the modified example of the first embodiment, the measurement position determination unit 301 may detect the specific part based on change, in terms of the longitudinal direction, in values for blood vessel diameter, blood vessel cross-sectional area, or the like, or based on derivatives (i.e., rates of change) of such values. Alternatively, the measurement position determination unit 301 may detect the specific part based on transverse cross-sectional shape of the blood vessel or based on a number of blood vessel cross-sections that are present. Note that the representative position of each segment may for example be a center point of the lumen, midway along the segment in terms of the longitudinal direction. FIG. 11A illustrates an example for determination of measurement positions in which measurement positions 641-645 are determined in segments 631-634. Note that the segments may correspond to parts of the carotid artery such as the common carotid artery and the carotid sinus, or may be determined as intervals of fixed length, arranged in order starting from the bifurcation.

The diseased part detection unit 302 judges, for each segment, whether blood flow velocity at the representative position exceeds a threshold value. When the blood flow velocity exceeds the threshold value, the corresponding segment is determined to be a disease candidate segment in which presence of a diseased part such as a plaque is suspected. At a position in a blood vessel at which a plaque is present, stenosis of the blood vessel causes increased blood flow velocity. Consequently, percentage stenosis of the blood vessel can be calculated based on blood flow velocity acquired through measurement by Doppler ultrasound. Percentage stenosis indicates a percentage of cross-sectional area of the blood vessel that is occupied by a plaque. For example, when maximum blood flow velocity in the common carotid artery exceeds 200 cm/s, percentage stenosis can be estimated to be at least 70%. Note that average blood flow velocity varies for different parts of the carotid artery such as the common carotid artery and the internal carotid artery. As a consequence, a threshold value for blood flow velocity may be set individually for each segment. Also, as an alternative to processing using a threshold value for blood flow velocity, a plurality of measurement positions may be determined in each segment in order to acquire a distribution of blood flow velocity in the segment. Judgment may subsequently be performed as to whether the distribution is uniform, and the segment may be determined to be a disease candidate segment when judging that the distribution is not uniform. FIG. 11B illustrates blood flow velocity at each of measurement positions 641-645 illustrated in FIG. 11A. When blood flow velocity at measurement position 641 in segment 631 exceeds a threshold value, stenosis is considered to be greater in segment 631 than in segments 632-634, and thus the diseased part detection unit 302 determines that segment 631 is a disease candidate segment.

The measurement information display unit 303 displays blood flow velocity for each segment, and also displays information for a disease candidate segment indicating that presence of a diseased part such as a plaque is suspected in the disease candidate segment. The information indicating that presence of a diseased part is suspected in the disease candidate segment may for example be displaying the disease candidate segment as a different color to other segments.

<Operation>

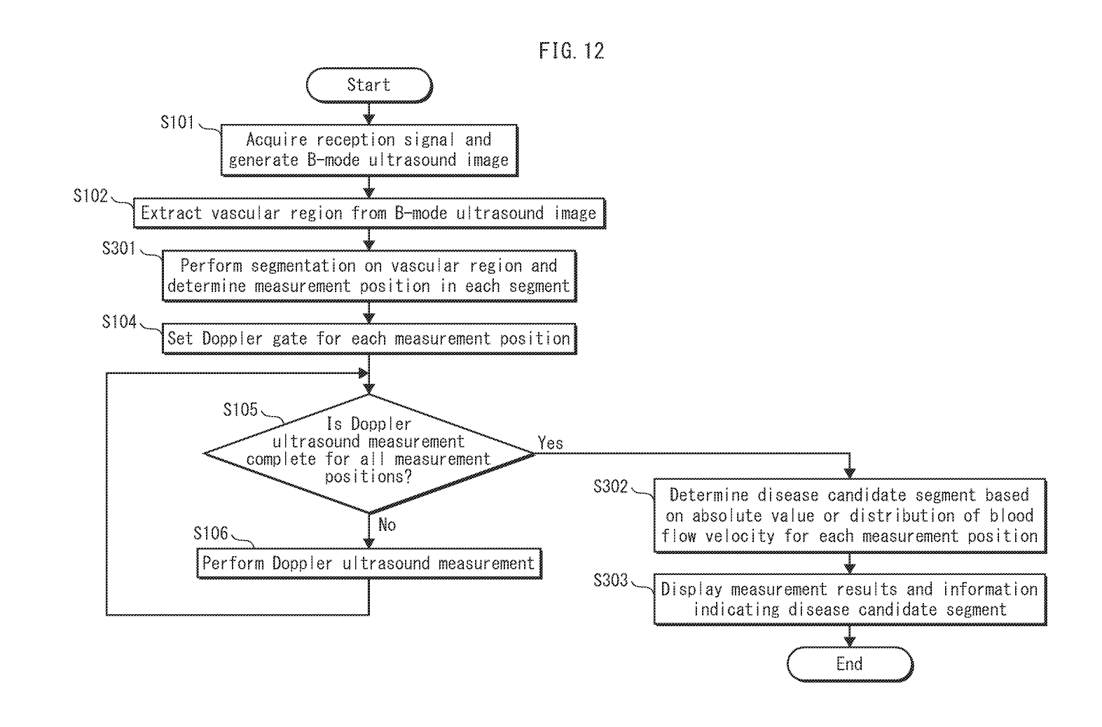

FIG. 12 is a flowchart illustrating operation of the ultrasound diagnostic apparatus 30. Steps in FIG. 12 which are the same as steps in FIG. 3 are indicated using the same reference signs and explanation thereof is omitted.

After the ultrasound diagnostic apparatus 30 has performed Step S102, the measurement position determination unit 301 detects the boundary between the common carotid artery and the carotid sinus, or the bifurcation of the carotid artery (i.e., the boundary between the carotid sinus and the internal and external carotid arteries) as a specific part of the vascular region, performs segmentation of the vascular region in accordance with the specific part, and determines a representative position in each of the segments to be a measurement position (Step S301).

Once the ultrasound diagnostic apparatus 30 has completed measurement by Doppler ultrasound for all of the measurement positions (Step S105: Yes), the diseased part detection unit 302 determines whether each of the segments is a disease candidate segment based on an absolute value of blood flow velocity or a distribution of blood flow velocity measured with respect to the corresponding measurement position (Step S302).

The measurement information display unit 303 subsequently displays measurement results and information indicating a diseased part (Step S303).

<Supplementary Explanation>

(1) In the first, second, and third embodiments the ultrasound probe is a linear probe. The present supplementary explanation explains operation in a configuration in which a matrix ultrasound probe is used, which includes a 2D array of ultrasound transducers and which can acquire an image of a 3D region below the ultrasound probe.

The matrix ultrasound probe can acquire reception signals for a plurality of scan planes having different positions or orientations, by controlling position of ultrasound transducers that are used in a certain scan or phase of an ultrasound signal emitted from the ultrasound transducers. Consequently, when the matrix ultrasound probe is used, searching for a longitudinal cross-sectional image of a blood vessel can be performed without changing position of the matrix ultrasound probe. Furthermore, even when the matrix ultrasound probe moves during measurement, a scan plane can be moved such that a longitudinal cross-sectional image can be rendered for position of the matrix ultrasound probe after movement thereof, and consequently it is not necessary to move the matrix ultrasound probe back to the original position thereof.

For example, a transverse cross-sectional image of the blood vessel may first be rendered using a scan plane parallel to the transverse direction and position of the blood vessel may be acquired through detection of a media-adventitia interface or the like in the transverse cross-sectional image. A longitudinal cross-sectional image can subsequently be found simply by switching to a scan plane parallel to the longitudinal direction at the aforementioned position. Preferably the longitudinal cross-sectional image which is rendered should be along a maximum effective plane. The term maximum effective plane refers to a scan plane passing centrally through the blood vessel. The maximum effective plane is a plane in which width of the blood vessel is a maximum value equivalent to diameter of the blood vessel. Therefore, when width of the blood vessel in the rendered longitudinal cross-sectional image changes when the ultrasound probe is moved, tracking for the maximum effective plane can be performed by changing position or orientation of the scan plane such that width of the blood vessel in the scan plane is the maximum value equivalent to diameter of the blood vessel. In the second embodiment, the ultrasound diagnostic apparatus 20 acquires position information for a scan plane using position sensors, but when a matrix ultrasound probe is used the position sensors can be omitted, and when generating a 3D image from a plurality of B-mode ultrasound images, correspondence between position information when generating the 3D image and position information when performing measurement by Doppler ultrasound can be easily realized.

(2) In the first, second, and third embodiments measurement by Doppler ultrasound is performed on the carotid artery, but the measurement target is not limited to a blood vessel such as the carotid artery, and may for example alternatively be the heart. In a situation in which the measurement target is the heart, a measurement position is determined by detecting, for example, a boundary between a ventricle and an atrium or a position of a valve. For example, determining a plurality of measurement positions in proximity to the valve and examining a distribution of blood flow velocities measured thereat, enables judgment as to whether the valve is moving normally.

### Fourth Embodiment

By recording on a recording medium such as a floppy disk, a program for implementing the control method for an ultrasound diagnostic apparatus described in each of the above embodiments, an independent computer system can easily execute processing described in each of the above embodiments.

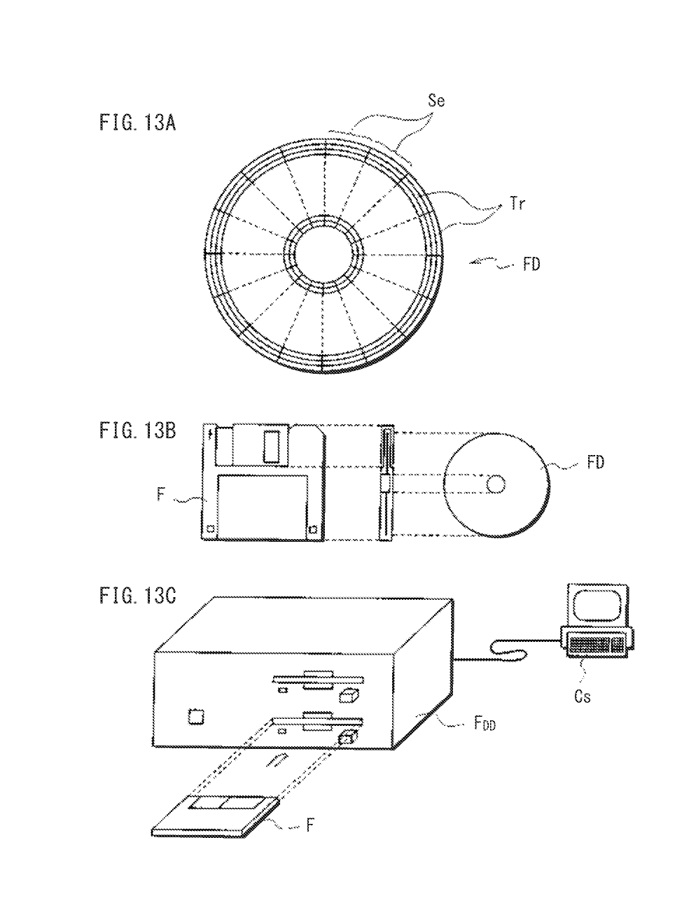

FIGS. 13A-13C are provided for explaining a configuration in which the control method for an ultrasound diagnostic apparatus described in each of the above embodiments is executed by a computer system using a program recorded on a recording medium such as a floppy disk.

FIG. 13A illustrates an example of physical format of a floppy disk FD which is an example of a recording medium. FIG. 13B illustrates an external front view, a cross-section view and an internal view of the floppy disk FD. The floppy disk FD is housed in a case F. A plurality of tracks Tr are formed on a surface of the floppy disk FD in concentric circles from an outer circumference to an inner circumference of the floppy disk FD. Each track is divided into 16 sectors Se in terms of angle from a center of the floppy disk FD. Therefore, the above program can be stored on a floppy disk by recording the program in an allotted region on the floppy disk FD.

FIG. 13C illustrates a configuration for executing the program recorded on the floppy disk FD. When recording the program for implementing the control method of an ultrasound diagnostic apparatus on the floppy disk FD, a computer system Cs writes the program using a floppy disk drive. Furthermore, when constructing, in a computer system, the control method of an ultrasound diagnostic apparatus which is implemented by the program recorded on the floppy disk, the program is read from the floppy disk by the floppy disk drive and is transmitted to the computer system.

In the above explanation, a floppy disk is used as an example of a recording medium, but alternatively an optical disk may be used in the same way. The recording medium is not limited to being a floppy disk or an optical disk, and alternatively may be any medium on which a program can be recorded, such as an IC (Integrated Circuit) card or a ROM (Read Only Memory) cassette.

Functional blocks in each of the ultrasound diagnostic apparatuses illustrated in FIGS. 1, 7, and 10 may typically be implemented through large scale integration (LSI) as integrated circuits. Each of the functional blocks may be integrated into a chip or alternatively all or a portion of the functional blocks may be integrated into a single chip.

The above refers to LSI, but depending on the degree of integration the above may also be referred to as IC, system LSI, super LSI or ultra LSI.

Also, circuit integration is not limited to LSI and may alternatively be realized through a dedicated circuit or a general processor. For example, a dedicated circuit for graphics processing may be used such as a graphic processing unit (GPU). A field programmable gate array (FPGA), which is programmable after the LSI is manufactured, or a reconfigurable processor, which allows for reconfiguration of the connection and setting of circuit cells inside the LSI, may alternatively be used.

Furthermore, if technology for forming integrated circuits that replaces LSI were to emerge, owing to advances in semiconductor technology or to another derivative technology, the integration of functional blocks may naturally be accomplished using such technology.

Furthermore, the configuration elements of each of the ultrasound diagnostic apparatuses illustrated in FIGS. 1, 7, and 10 may be connected via a network such as the Internet or a local area network (LAN). For example, in an alternative configuration an ultrasound image may be read from an accumulation device or a server in a network, which stores the ultrasound image therein. Additional functions of each of the configuration elements may also be performed through the network.

### Fifth Embodiment

The present embodiment explains a method used by the 3D shape analysis unit 201 for detecting a specific part from a plurality of transverse cross-sectional images (B-mode ultrasound images) that are successively captured of a blood vessel at different positions in the longitudinal direction, a method used by the Doppler gate setting unit 105 for setting a Doppler gate, and a method for assisting movement of the ultrasound probe.

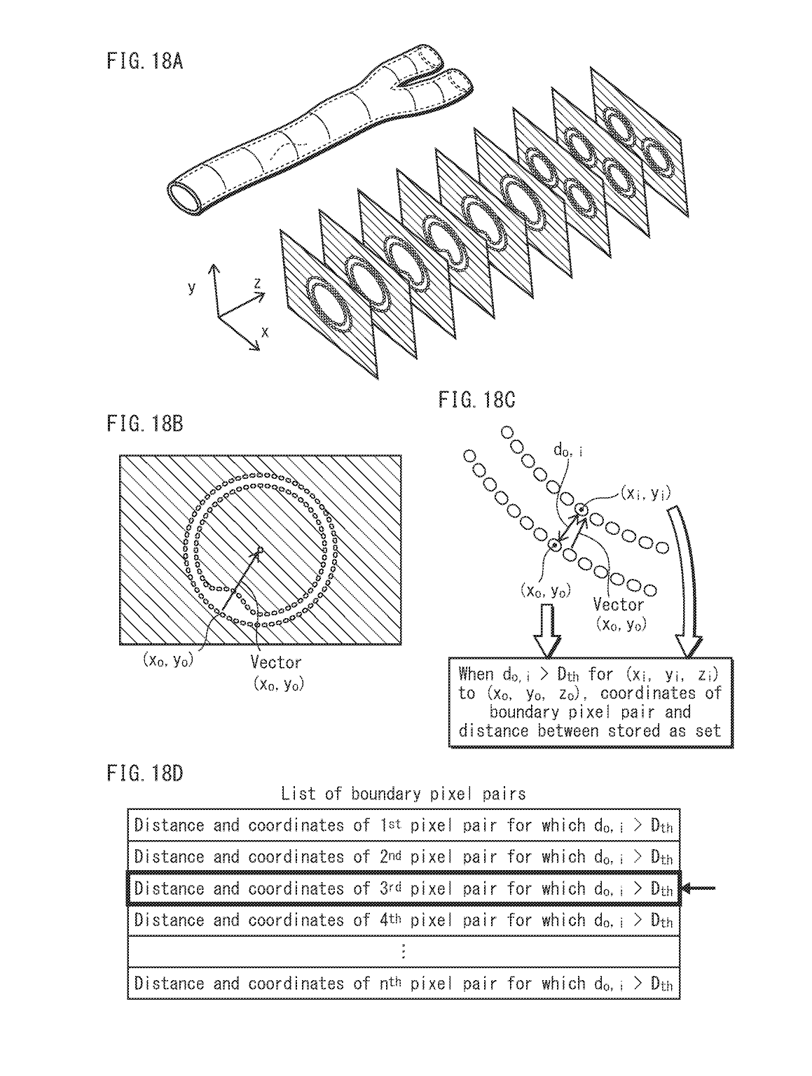

FIG. 18A illustrates a carotid artery and a plurality of transverse cross-sectional images of the carotid artery that are successively acquired at different positions in the longitudinal direction. Coordinates of each pixel in each of the transverse cross-sectional images are expressed using an xyz 3D coordinate system in which the x-axis is a horizontal axis of each of the images, the y-axis is a vertical axis of each of the images, and the z-axis is the longitudinal direction of the carotid artery. Note that the y-axis is approximately perpendicular to surface of the skin. Each of the transverse cross-sectional images is configured by pixel value data for M pixels horizontally by N pixels vertically. The pixel value data indicates a brightness scale of the pixels through bit values of predetermined length. Each of the transverse cross-sectional images is a scan of a subject, and thus inter-pixel separation in the transverse cross-sectional image is proportional to dimensions of the subject which is the measurement target. Consequently, actual dimensions of the subject can be calculated by multiplying actual size per one pixel by the inter-pixel separation. The following explanation assumes that inter-pixel separation is equivalent to actual dimensions of the subject.

FIG. 18B illustrates a transverse cross-sectional image. As explained in the first embodiment, edge detection processing or the like can be performed on the transverse cross-sectional image in order to detect contours of the vascular wall as a double loop. The double loop consists of an outer pixel loop composed of a plurality of pixels, and an inner pixel loop composed of a plurality of pixels. The outer pixel loop indicates a media-adventitia interface and the inner pixel loop indicates a lumen-intima interface. The 3D shape analysis unit 201 selects the outer pixel loop as a target for processing. In the present example, a pixel selected as a target for processing has coordinates (xₒ, yₒ).

In FIG. 18B, a vector (xₒ, yₒ) is a vector from coordinates (xₒ, yₒ) to a center point of the double loop. When selecting a pixel, the vector (xₒ, yₒ) is defined and a search is performed along the vector (xₒ, yₒ) for a pixel of the inner pixel loop.

FIG. 18C illustrates that during the aforementioned search, a pixel of the inner pixel loop which is found has coordinates (xᵢ, yᵢ). Next, inter-pixel distance d_{o,i} between the pixels at coordinates (xₒ, yₒ) and (xᵢ, yᵢ) is calculated. Inter-pixel distance d_{o,i} is equivalent to IMT. A position at which IMT exceeds a fixed value is defined as a plaque. Therefore, when the 3D shape analysis unit 201 judges that inter-pixel distance d_{o,i}, exceeds the fixed value, the 3D shape analysis unit 201 stores coordinates (xₒ, yₒ), coordinates (xᵢ, yᵢ), and inter-pixel distance d_{o,i}, as a set.

The processing described above is repeated for each pixel of the outer pixel loop. Through the above, a set of coordinates (xₒ, yₒ), coordinates (xᵢ, yᵢ), and inter-pixel distance d_{o,i}, is stored in a list for each position in a single transverse cross-sectional image at which IMT exceeds the fixed value. FIG. 18D illustrates the aforementioned list. A position at which IMT is greatest in the transverse cross-sectional image can be determined by searching for coordinates (xₒ, yₒ) included in the same set as a greatest inter-pixel distance d_{o,i} in the list. Furthermore, a position of maximum hypertrophy in the 3D image can be determined by comparing respective greatest inter-pixel distances d_{o,i} of the plurality of transverse cross-sectional images.

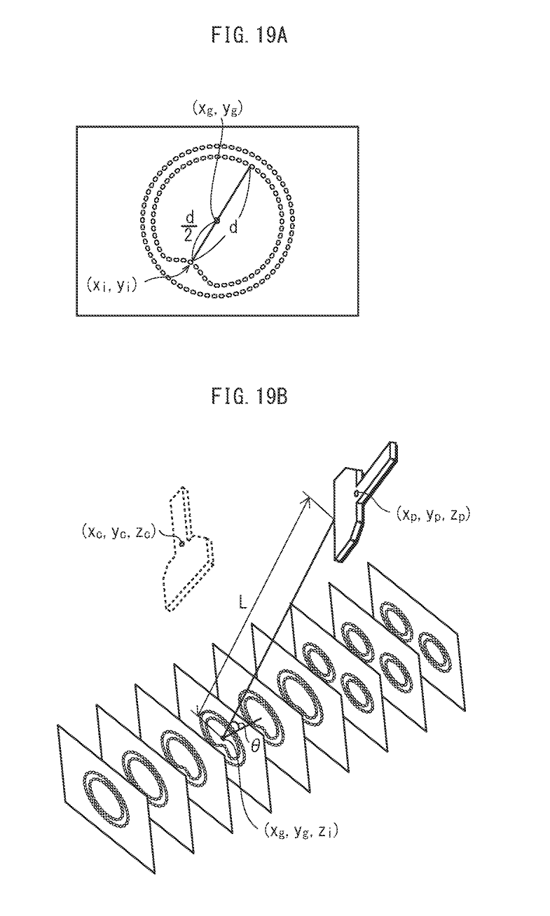

The following explains, with reference to FIG. 19A, setting of a Doppler gate using 3D coordinates of the position of maximum hypertrophy. Suppose that at the position of maximum hypertrophy the tunica intima has coordinates (xᵢ, yᵢ, zᵢ), and that diameter d of the tunica intima is a line from the coordinates (xᵢ, yᵢ, zᵢ), passing through a center point of the blood vessel cross-section to the tunica intima at an opposite side of the lumen. A center point of the Doppler gate is set at coordinates (x_{g}, y_{g}, zᵢ), a distance d/2 in a direction passing through the center point of the blood vessel cross-section from coordinates (xᵢ, yᵢ, zᵢ).

Consequently, the ultrasound probe should be positioned on a straight line that passes through the center point (x_{g}, y_{g}, zᵢ) of the Doppler gate and that forms an angle θ with respect to the z-axis in a plane parallel to the y-axis. FIG. 19B illustrates the ultrasound probe positioned at coordinates (xₚ, yₚ, zₚ) on a straight line such as described above, separated from the center point (x_{g}, y_{g}, zᵢ) of the Doppler gate by a distance L.

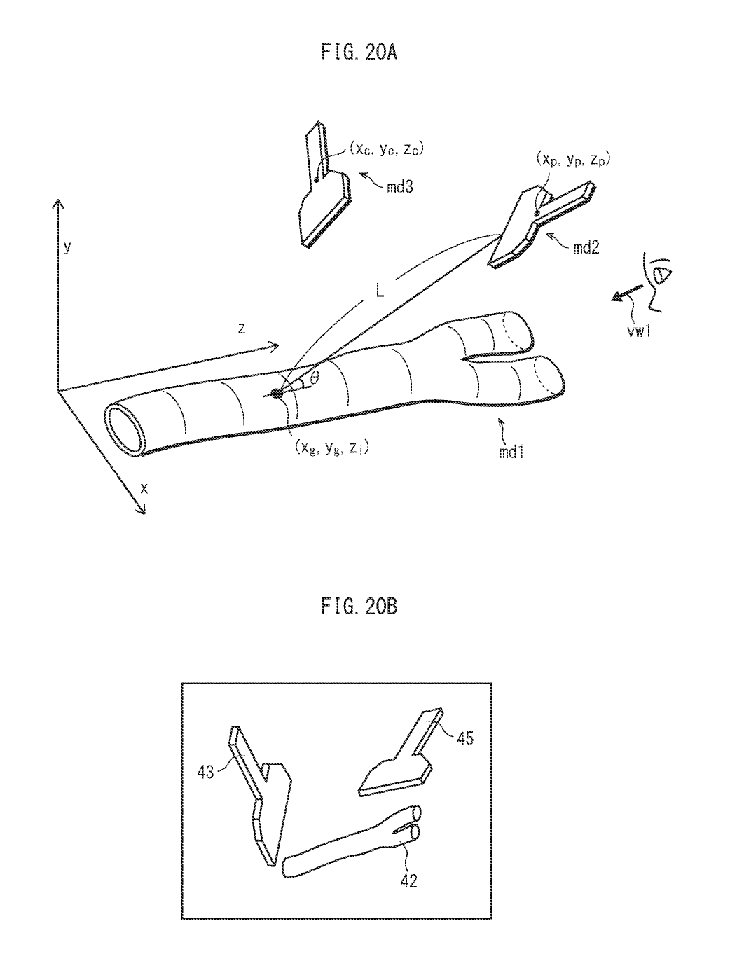

When a current position of the ultrasound probe detected by the position information acquisition unit 202 has coordinates (x_{c}, y_{c}, z_{c}), guidance of the ultrasound probe to coordinates (xₚ, yₚ, zₚ) is required. FIG. 20B illustrates an assistance image for guiding the ultrasound probe. In order to create the assistance image, it is necessary to display a 3D model of the vascular region, and to display a 3D model of the ultrasound probe at both coordinates (xₚ, yₚ, zₚ) and coordinates (x_{c}, y_{c}, z_{c}). The 3D models are data that define external shape through relative arrangement of 3D coordinates. The vascular region and the ultrasound probe are each displayed as a polyhedron through interpolation of a 3D image configuring the 3D model.

FIG. 20A illustrates the 3D models arranged in a 3D space. 3D Model md1 is a 3D model of the vascular region. The 3D model md1 is created by using coordinates of pixel loops in the plurality of transverse cross-sectional images as 3D coordinates defining external shape of the 3D model md1, and by performing interpolation using surface shape. 3D model md2 is a 3D model indicating measurement position and measurement orientation of the ultrasound probe. Using 3D coordinates included in CAD data for the ultrasound probe which is stored in advance, the 3D model md2 is positioned at coordinates (xₚ, yₚ, zₚ) and is orientated towards the center point (x_{g}, y_{g}, zᵢ) of the Doppler gate. 3D model md3 is a 3D model indicating current position and current orientation of the ultrasound probe. Using 3D coordinates included in CAD data for the ultrasound probe which is stored in advance, the 3D model md3 is positioned at coordinates (x_{c}, y_{c}, z_{c}) and is orientated using current orientation of the ultrasound probe. Vector vw1 is a vector defining a theoretical viewpoint and viewing direction in the 3D space. A viewport is arranged in the 3D space in accordance with the theoretical viewpoint and viewing direction. By performing rendering processing with respect to the 3D models arranged in the 3D space, a projected image of the 3D space can be obtained in the corresponding viewport plane. The rendering processing is a series of processing operations including coordinate conversion, illumination calculation, texture mapping, and viewport conversion. In the coordinate conversion, 3D coordinates configuring the 3D models are converted from a local coordinate system to a global coordinate system. In the illumination calculation, illumination of a surface of each of the 3D models is calculated in accordance with position of a light source in the 3D space. In the texture mapping, image data is pasted to a surface of each of the 3D models in order to achieve a realistic appearance.

FIG. 20B illustrates the projected image which is created through rendering performed with respect to the 3D space. An assistance image is generated by performing further modification on the projected image in order to form a composite image with a scan plane, path information, or the like.

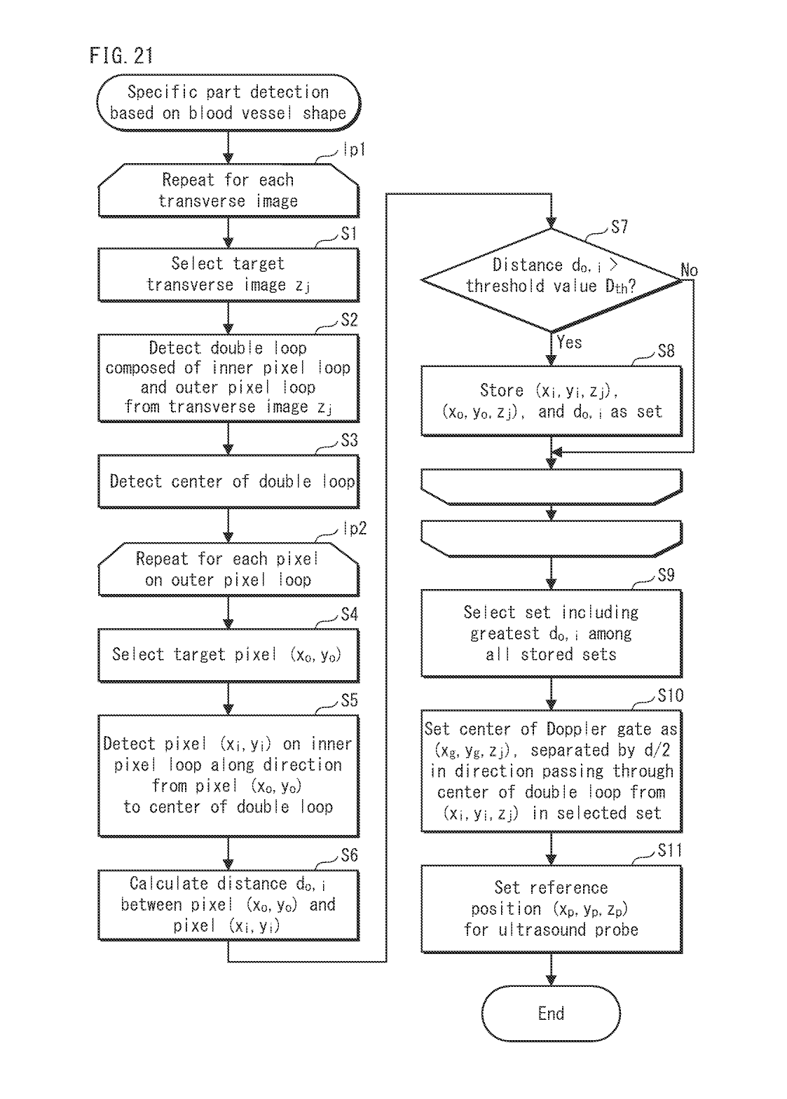

The above operations can be generalized as processing with respect to hardware such as a CPU (Central Processing Unit) or a memory. FIG. 21 is a flowchart illustrating a process order of detecting a specific part from a plurality of transverse cross-sectional images captured of a vascular region. The process includes a processing loop lp1 which is repeated for each transverse cross-sectional image and a processing loop lp2 which is repeated for each pixel of an outer pixel loop of a double loop. In the processing loop lp1, a target transverse cross-sectional image zⱼ is selected (Step S1), a double loop is detected from the transverse cross-sectional image zⱼ (Step S2), and a center point of the double loop is detected (Step S3). After completion of the above steps the processing loop lp2 is executed. In the processing loop lp2, a target pixel (xₒ, yₒ) is selected from an outer pixel loop (Step S4), a pixel (xᵢ, yᵢ) of an inner pixel loop located along a direction towards the center point of the double loop from the pixel (xₒ, yₒ) is detected (Step S5), and inter-pixel distance d_{o,i} between the pixel (xₒ, yₒ) and the pixel (xᵢ, yᵢ) is calculated (Step S6). In the processing loop lp2, judgment is subsequently performed as to whether inter-pixel distance d_{o,i} exceeds a threshold value Dₜₕ (Step S7), and when judging affirmatively (xᵢ, yᵢ, zⱼ), (xₒ, yₒ, zⱼ), and d_{o,i} are stored as a set (Step S8). The processing described above is repeated for each pixel of the outer pixel loop as a target of the processing. After processing loops lp1 and lp2 have been completed, a set including a greatest inter-pixel distance d_{o,i} is selected (Step S9), coordinates (x_{g}, y_{g}, zⱼ), which are coordinates d/2 in a direction passing through the center point of the double loop from the coordinates (xᵢ, yᵢ, zⱼ) included in the selected set, are set as a center point of a Doppler gate (Step S10), and a reference position (xₚ, yₚ, zₚ) is set for the ultrasound probe (Step S11).

Through the present embodiment, an appropriate assistance image can be generated for guiding the ultrasound probe to a position suitable for measuring blood flow velocity in proximity to a plaque. The assistance image can be generated as described above, by performing image processing on a plurality of successively acquired transverse cross-sectional images of a blood vessel, and by performing 3D computer graphics processing.

<Supplementary Explanation>

(1) In the fifth embodiment, a pixel (xᵢ, yᵢ) of the inner pixel loop is detected which is present along the direction between the pixel (xₒ, yₒ) and the center point of the double loop, and inter-pixel distance d_{o,i} between the pixel (xₒ, yₒ) and the pixel (xᵢ, yᵢ) is calculated, but alternatively inter-pixel distance d_{o,i} may be calculated as described below. For example, all pixels (xᵢ, yᵢ) of the inner pixel loop for which an x-coordinate and a y-coordinate respectively differ from an x-coordinate and a y-coordinate of the pixel (xₒ, yₒ) by no greater than a predetermined value may be detected, and a smallest distance between any one of the pixels (xᵢ, yᵢ) and the pixel (xₒ, yₒ) may be set as inter-pixel distance d_{o,i}.

(2) In the fifth embodiment, the z-axis, which corresponds to the longitudinal direction, is equivalent to the blood vessel extension direction, but alternatively a center point of the outer pixel loop of the double loop may be detected for each of the transverse cross-sectional images zⱼ, and a polygonal line connecting the center points, or a straight line approximately connecting the center points, may be used as the blood vessel extension direction. Through the above configuration, the present invention can be implemented even in a situation in which the transverse cross-sectional images zⱼ are not perpendicular to the blood vessel extension direction, and thus in which the z-axis is not equivalent to the blood vessel extension direction.

### Sixth Embodiment

The present embodiment explains a method for performing segmentation of a vascular region through image processing.

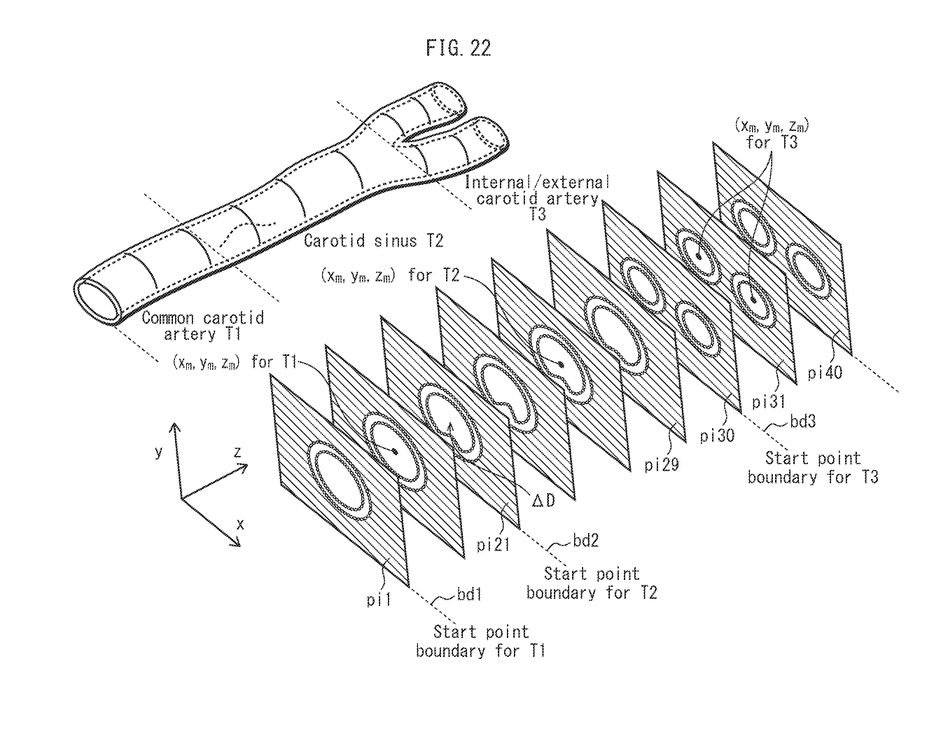

FIG. 22 illustrates a vascular region which is a target for segmentation and a plurality of transverse cross-sectional images pi1-pi40 which are targets for image processing. A z-coordinate in a 3D space is assigned to each of the plurality of transverse cross-sectional images in the bottom-right of FIG. 22. The z-coordinate corresponds to position of a body part on which a scan is performed using the ultrasound probe, and by performing a count of the z-coordinates, an approximate dimension of the body part in the longitudinal direction can be estimated.

The z-coordinate assigned to each of the plurality of transverse cross-sectional images is in accordance with actual size of the body part on which the scan is performed. A vascular region illustrated in the top-left of FIG. 22 is configured by three segments respectively corresponding to a common carotid artery, a carotid sinus, and internal and external carotid arteries. Among the plurality of transverse cross-sectional images, transverse cross-sectional image pi21 is an image for which amount of change in diameter of the blood vessel, indicated by inter-pixel distance between an outer pixel loop and an inner pixel loop of a double loop, exceeds a predetermined threshold value. As explained further above, amount of change in diameter of the carotid artery differs in the carotid sinus compared to the common carotid artery. Therefore, a transverse cross-sectional image for which amount of change in diameter of the double loop exceeds the predetermined threshold value is defined as a boundary between the common carotid artery and the carotid sinus. Consequently, a position corresponding to transverse cross-sectional image pi21 is a start point of the carotid sinus, and is also a boundary between the carotid sinus and the common carotid artery which is located directly before the boundary.

On the other hand, note that two double loops are present in transverse cross-sectional image pi30. The presence of two double loops indicates that two blood vessels are present in the scan plane, and consequently indicates that both the internal carotid artery and the external carotid artery have been scanned in transverse cross-sectional image pi30. Only one double loop is present in transverse cross-sectional image pi29 directly before transverse cross-sectional image pi30, and thus transverse cross-sectional image pi30 is defined as a start point of the internal and external carotid arteries.

By defining boundaries of the segments in terms of z-coordinates as described above, an extent of each of the segments in terms of z-coordinates is clarified. Note that bd1 and bd2 are respective z-coordinates of transverse cross-sectional images pi21 and pi30, and are used as boundaries for partitioning the vascular region into segments. In FIG. 22, segments T1, T2 and T3 are three segments which are obtained when performing segmentation using bd1 and bd2 as start points. Transverse cross-sectional image pi1 is defined as a start point of segment T1 and transverse cross-sectional image pi40 is defined as an end point of segment T3. Segment T1 corresponds to the common carotid artery, segment T2 corresponds to the carotid sinus, and segment T3 corresponds to the internal and external carotid arteries. Note that coordinates (xₘ, yₘ, zₘ) in each of the segments are measurement coordinates which are set centrally in the corresponding segment.

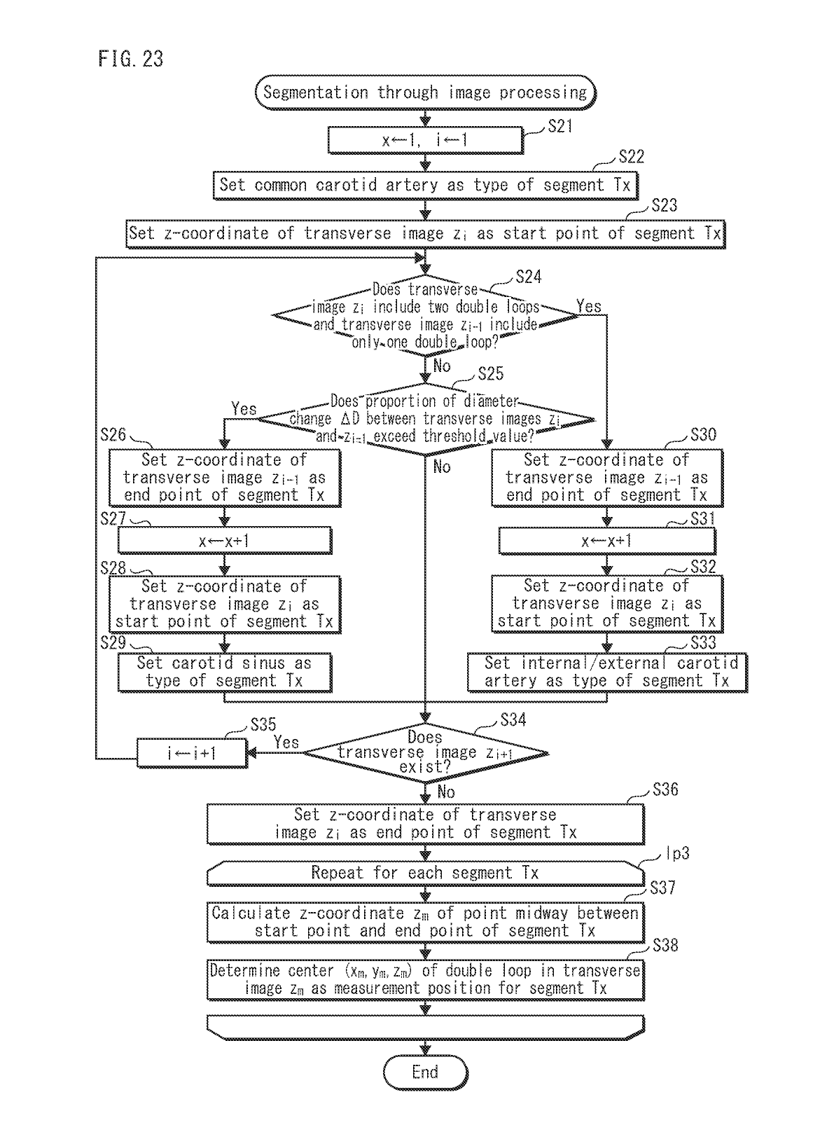

FIG. 23 illustrates order of processing for acquiring segments T1, T2 and T3. Transverse cross-sectional image zᵢ is a transverse cross-sectional image which is processing target and which is defined by a z-coordinate thereof. Segment Tx is a segment which is generated. More specifically, each segment Tx is one of segments T1, T2 and T3.

First, variables x and i are initialized (Step S21).

Next, a type of segment Tx (=T1) is initially set as common carotid artery (Step S22).

Processing is subsequently performed in order to detect a segment boundary. First a z-coordinate of transverse cross-sectional image zᵢ is set as a start point of segment Tx (Step S23). Next judgment is performed as to whether a bifurcation has occurred by judging whether two double loops are present in transverse cross-sectional image zᵢ but only one double loop is present in transverse cross-sectional image zᵢ₋₁ (Step S24). When judging that a bifurcation has occurred, a bifurcation structure can be judged to be present between transverse cross-sectional image zᵢ₋₁ and transverse cross-sectional image zᵢ, and consequently a z-coordinate of transverse cross-sectional image zᵢ₋₁ is set as an end point of segment Tx (Step S30). Next, variable x is incremented (Step S31), a z-coordinate of transverse cross-sectional image zᵢ is set as a start point of segment Tx (Step S32), and a type of segment Tx is set as internal/external carotid artery (Step S33).

On the other hand, when a bifurcation structure is not detected in Step S24, a judgment is subsequently performed at to whether a proportion of diameter change ΔD between transverse cross-sectional image zᵢ₋₁ and transverse cross-sectional image zᵢ exceeds a predetermined threshold value (Step S25). When the proportion of diameter change ΔD exceeds the threshold value, a boundary between a common carotid artery and a carotid sinus is judged to be present between transverse cross-sectional image zᵢ₋₁ and transverse cross-sectional image zᵢ, and thus a z-coordinate of transverse cross-sectional image zᵢ₋₁ is set as end point of segment Tx in the same way as described for Step S30 (Step S26). Next, variable x is incremented (Step S27), a z-coordinate of transverse cross-sectional image zᵢ is set as a start point of segment Tx (Step S28), and a type of segment Tx is set as carotid sinus (Step S29).

Next, judgment is performed as to whether transverse cross-sectional image zᵢ₊₁ exists (Step S34). When judging affirmatively in Step S34, variable i is incremented (Step S35), and boundary detection processing is repeated from Step S24. When judging negatively in Step S34, processing has been completed for each transverse cross-sectional image zᵢ, and a z-coordinate of a last transverse cross-sectional image zᵢ to be processed is set as an end point of a segment Tx which is currently being processed (Step S36).

Once a start point and an end point have been determined for each of the segments, which respectively correspond to the common carotid artery, the carotid sinus, and the internal and external carotid arteries, a processing loop lp3 is executed in order to calculate a representative point for each of the segments. For example, for each segment Tx a z-coordinate zₘ is calculated which is midway between a z-coordinate of a start point and a z-coordinate of an end point of the segment Tx (Step S37), and subsequently a center point (xₘ, yₘ, zₘ) of the double loop at the z-coordinate zₘ is determined to be a measurement position with respect to segment Tx (Step S38).

Through the above processing, a measurement position is calculated, and thus determined, for each segment. A Doppler gate is set with respect to the measurement position, and in a situation in which measurement cannot be performed in accordance with the Doppler gate which is set, an assistance image is generated in order to guide the user to move the ultrasound probe to a position at which measurement can be performed in accordance with the Doppler gate.

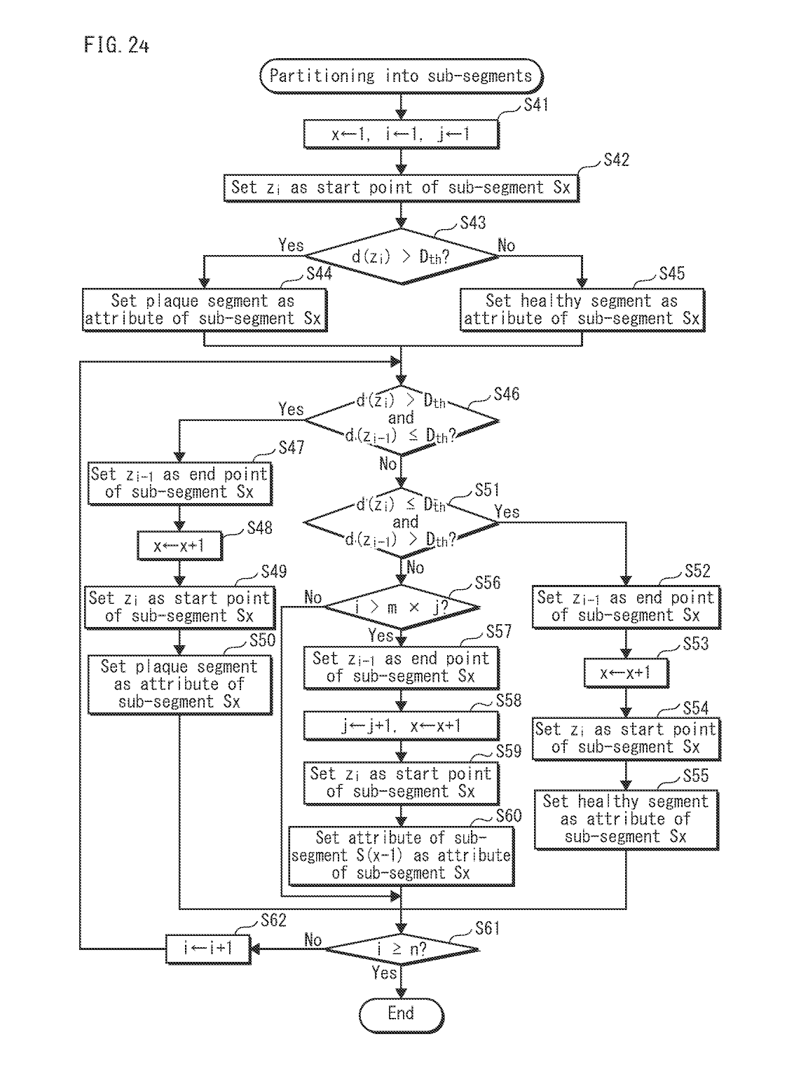

FIG. 24 illustrates an order of processing for partitioning each of the segments, corresponding respectively to the common carotid artery, the carotid sinus, and the internal and external carotid arteries, into a plurality of sub-segments Sx in accordance with whether or not a plaque is present. In the processing illustrated in FIG. 24, n (n is an integer greater than 1) indicates a number of transverse cross-sectional images included in segment Tx which is a target for partitioning into sub-segments, and m (m is an integer no greater than n) indicates a maximum number of transverse cross-sectional images in each sub-segment Sx.

First, variables x, i, and j are initialized (Step S41). Next, zᵢ is set as a start point of sub-segment Sx (=S1) (Step S42). A judgment is subsequently performed as to whether IMT d(zᵢ) at zᵢ exceeds a predetermined threshold value Dₜₕ (Step S43). IMT d(zᵢ) is calculated in the same way as in the fifth embodiment as a distance between a pixel on an outer pixel loop and a pixel on an inner pixel loop of a double loop in transverse cross-sectional image zᵢ.

When the IMT d(zᵢ) exceeds the threshold value Dₜₕ, “plaque segment” is set as an attribute of sub-segment Sx (Step S44), and when the IMT d(zᵢ) does not exceed the threshold value Dₜₕ, “healthy segment” is set as an attribute of sub-segment Sx (Step S45).

Next, a judgment is performed as to whether IMT d(zᵢ) at zᵢ exceeds the threshold value Dₜₕ but IMT d(zᵢ₋₁) at zᵢ₋₁ does not exceed the threshold value Dₜₕ (Step S46). An affirmative judgment in Step S46 indicates that between transverse cross-sectional image zᵢ₋₁ and transverse cross-sectional image zᵢ, there is a change from a region in which a plaque is not present to a region in which a plaque is present. Therefore, when judging affirmatively in Step S46, zᵢ₋₁ is set as an end point of sub-segment Sx (Step S47), variable x is incremented (Step S48), zᵢ is set as a start point of a new sub-segment Sx (Step S49), and “plaque segment” is set as an attribute of the new sub-segment Sx (Step S50).

When judging negatively in Step S46, a judgment is performed as to whether the IMT d(zᵢ) at zᵢ does not exceed the threshold value Dₜₕ but IMT d(zᵢ₋₁ at zᵢ₋₁ does exceed the threshold value Dₜₕ (Step SM). An affirmative judgment in Step SM indicates that between transverse cross-sectional image zᵢ₋₁ and transverse cross-sectional image zᵢ, there is a change from a region in which a plaque is present to a region in which a plaque is not present. Therefore, when judging affirmatively in Step SM, zᵢ₋₁ is set as an end point of sub-segment Sx (Step S52), variable x is incremented (Step S53), zᵢ is set as a start point of a new sub-segment Sx (Step S54), and “healthy segment” is set as an attribute of the new sub-segment Sx (Step S55).

On the other hand, negative judgments in both of Steps S46 and S51 indicates that between transverse cross-sectional image zᵢ₋₁ and transverse cross-sectional image zᵢ, there is no change in terms of presence or absence of a plaque. In such a situation, a judgment is performed as to whether variable i exceeds m×j (Step S56), due to m indicating a maximum number of transverse cross-sectional images in a sub-segment. When variable i exceeds m×j, zᵢ₋₁ is set as an end point of sub-segment Sx (Step S57), variables j and x are incremented (Step S58), and zᵢ is set as a start point of a new sub-segment Sx (Step S59). In the situation described above, no change occurs in terms of presence or absence of a plaque between transverse cross-sectional image zᵢ₋₁ and transverse cross-sectional image zᵢ, and consequently an attribute of the new sub-segment Sx is set as the same as an attribute of sub-segment S(x−1) (Step S60).

Next, a judgment is performed as to whether variable i is at least equal to n (Step S61). When judging negatively in Step S61, variable i is incremented (Step S62), and processing in repeated from Step S46 in order that the processing described above is performed for each transverse cross-sectional image.

Through the above, segment Tx can be partitioned into a plurality of sub-segments Sx in accordance with whether or not a plaque is present, and such that each of the sub-segments Sx is no greater than a predetermined length. By for example performing the processing loop lp3 described above with respect to each sub-segment Sx, a measurement position can be determined in accordance with each plaque segment and each healthy segment. Consequently, blood flow velocity can be easily measured with respect to each plaque segment and each healthy segment.

### Other Modified Examples Relating to the Embodiments

(1) In the first to fifth embodiments, the ultrasound image acquisition unit 101 generates a B-mode ultrasound image from a reception signal which is acquired by scanning a measurement target using the ultrasound probe, and the ultrasound image acquisition unit 101 outputs the B-mode ultrasound image to the vascular region detection unit 102, but the above is not a limitation on the present invention. For example, alternatively the ultrasound image acquisition unit 101 may output color mode information or the like based on the reception signal, or may acquire an image which has been generated by an external image generation device. Through the above, the ultrasound image acquired by the ultrasound image acquisition unit 101 is not limited to being a B-mode ultrasound image. Furthermore, the ultrasound image acquisition unit 101 is not limited to using an image acquired through ultrasonography, and may alternatively use a different type of image.

Further alternatively, the ultrasound image acquisition unit 101 may acquire the reception signal from scanning of the measurement target using the ultrasound probe, and may output the reception signal to the vascular region detection unit 102 without modification. In such a configuration, units such as the vascular region detection unit 102, the measurement position determination unit 103, and the Doppler gate setting unit 104 may use the reception signal without modification and thus without conversion to an image.

(Supplementary Explanation)

The following explains various configurations and effects of an ultrasound diagnostic apparatus and a control method thereof relating the present invention.

(1) One aspect of the present invention is an ultrasound diagnostic apparatus for measuring blood flow velocity by emitting ultrasound towards a measurement target via an ultrasound probe and by receiving a reflected wave via the ultrasound probe, the ultrasound diagnostic apparatus comprising: an ultrasound image acquisition unit configured to acquire an ultrasound image which has been captured of the measurement target; a vascular region detection unit configured to detect, from the ultrasound image, a vascular region corresponding to a blood vessel in the measurement target; a measurement position determination unit configured to detect a specific part of the vascular region based on shape of the vascular region and to determine, in accordance with the specific part, one or more measurement positions in the vascular region in terms of longitudinal direction thereof; and a Doppler gate setting unit configured to set, with respect to each of the measurement positions, a Doppler gate in a lumen region of the vascular region.

Another aspect of the present invention is a control method for an ultrasound diagnostic apparatus for measuring blood flow velocity by emitting ultrasound towards a measurement target via an ultrasound probe and by receiving a reflected wave via the ultrasound probe, the control method comprising: acquiring an ultrasound image which has been captured of the measurement target; detecting, from the ultrasound image, a vascular region corresponding to a blood vessel in the measurement target; detecting a specific part of the vascular region based on shape of the vascular region and determining, in accordance with the specific part, one or more measurement positions in the vascular region in terms of longitudinal direction thereof; and setting, with respect to each of the measurement positions, a Doppler gate in a lumen region of the vascular region.

Another aspect of the present invention is a non-transitory computer readable recording medium storing therein a program for causing a processor used in an ultrasound diagnostic apparatus to perform control processing comprising: acquiring an ultrasound image which has been captured of a measurement target; detecting, from the ultrasound image, a vascular region corresponding to a blood vessel in the measurement target; detecting a specific part of the vascular region based on shape of the vascular region and determining, in accordance with the specific part, one or more measurement positions in the vascular region in terms of longitudinal direction thereof; and setting, with respect to each of the measurement positions, a Doppler gate in a lumen region of the vascular region.

Through the above configuration, the ultrasound diagnostic apparatus relating to the one aspect of the present invention detects the specific part, which for example corresponds to a diseased part or an examination target in the measurement target, based on shape of the vascular region, automatically determines the one or more measurement positions in terms of the longitudinal direction of the blood vessel, and sets an appropriate Doppler gate with respect to each of the measurement positions. Therefore, the ultrasound diagnostic apparatus relating to the one aspect of the present invention enables simple and accurate measurement of blood flow velocity.

(2) Alternatively, in the ultrasound diagnostic apparatus of section (1), the vascular region detection unit may detect a vascular wall region of the vascular region during detection of the vascular region, and the measurement position determination unit may detect the specific part based on shape of the vascular wall region.

Through the above configuration, Doppler ultrasound measurement can be easily performed based on shape of the vascular wall region.

(3) Alternatively, in the ultrasound diagnostic apparatus of section (2), the specific part detected by the measurement position determination unit may be a part of the vascular region in which thickness of the vascular wall region exceeds a predetermined value.

Through the above configuration, Doppler ultrasound measurement can be easily performed based on a plaque.

(4) Alternatively, in the ultrasound diagnostic apparatus of section (3), the measurement position determination unit may determine each of the measurement positions to be in proximity to the specific part.

Through the above configuration, blood flow velocity can be easily and reliably measured in proximity to a plaque.

(5) Alternatively, in the ultrasound diagnostic apparatus of section (1), the measurement position determination unit may detect the specific part based on a change in shape of the vascular region in terms of the longitudinal direction, may perform segmentation of the vascular region in accordance with the specific part, and may determine a measurement position in each segment generated by the segmentation.

Through the above configuration, segmentation can be performed based on properties of the vascular region in terms of the longitudinal direction, and Doppler ultrasound measurement can be performed with respect to each of the segments resulting therefrom.

(6) Alternatively, in the ultrasound diagnostic apparatus of section (5), the change in shape of the vascular region may correspond to a change in diameter of the blood vessel, a change in a vascular wall of the blood vessel, or a bifurcation of the blood vessel.

Through the above configuration, segmentation of the vascular region can be performed based on structural properties of the blood vessel, and Doppler ultrasound measurement can be performed with respect to each of the segments resulting therefrom.

(7) Alternatively, in the ultrasound diagnostic apparatus of section (6), the vascular region may correspond to a carotid artery in the measurement target, and the specific part detected by the measurement position determination unit may correspond to a boundary between a common carotid artery and a carotid sinus, or to a boundary between the carotid sinus, and an internal carotid artery and an external carotid artery, in the measurement target.

Through the above configuration, Doppler ultrasound measurement of the carotid artery can be performed based on structure thereof.

(8) Alternatively, in the ultrasound diagnostic apparatus of section (6), the measurement position determination unit may determine each of the measurement positions to be in proximity to a center point of a corresponding one of the segments.

Through the above configuration, a representative position for performing Doppler ultrasound measurement can be easily determined for each of the segments.

(9) Alternatively, in the ultrasound diagnostic apparatus of section (1), the ultrasound image acquisition unit may acquire the ultrasound image of the measurement target via the ultrasound probe.

Through the above configuration, Doppler ultrasound measurement can be performed based on a longitudinal cross-section or the like of the blood vessel.

(10) Alternatively, the ultrasound diagnostic apparatus of section (1) may further comprise a probe position detection unit configured to detect position of the ultrasound probe and orientation of a scan plane of the ultrasound probe, wherein the ultrasound image acquisition unit may acquire a plurality of ultrasound images, captured of the measurement target along different scan planes of the ultrasound probe, and may generate a three dimensional image from the plurality of ultrasound images, and the vascular region detection unit may detect the vascular region from the three dimensional image.

Through the above configuration, a 3D image of the blood vessel can be generated using an acquisition unit that acquires 2D images, and Doppler ultrasound measurement can be performed based on 3D shape of the blood vessel.

(11) Alternatively, the ultrasound diagnostic apparatus of section (10) may further comprise a display unit configured to display the position of the ultrasound probe and the orientation of the scan plane which are detected, superposed on the three dimensional image, and to further display superposed information for guiding the ultrasound probe to a position for measuring blood flow velocity in accordance with the Doppler gate set by the Doppler gate setting unit.

Through the above configuration, the ultrasound probe can be guided to an appropriate position for performing Doppler ultrasound measurement.

(12) Alternatively, the ultrasound diagnostic apparatus of section (1) may further comprise a vascular region comparison unit, wherein the ultrasound image acquisition unit may acquire a first image and a second image that differ in terms of capture time, the vascular region detection unit may detect a first vascular region from the first image and a second vascular region from the second image, the vascular region comparison unit may compare the first vascular region to the second vascular region and judge whether a difference therebetween exceeds a predetermined threshold, and when the vascular region comparison unit judges affirmatively, the position determination unit may not determine a measurement position and the ultrasound image acquisition unit may acquire a new ultrasound image.

Through the above configuration, performance of Doppler ultrasound measurement can be prevented when position of the ultrasound probe is not constant, and consequently reliable results can be obtained.

Although the present invention has been fully described by way of examples with reference to the accompanying drawings, it is to be noted that various changes and modifications will be apparent to those skilled in the art. Therefore, unless such changes and modifications depart from the scope of the present invention, they should be construed as being included therein.

### INDUSTRIAL APPLICABILITY

An ultrasound diagnostic apparatus, and a control method thereof, relating to the present invention detects position of a plaque or other specific part of a blood vessel and automatically sets a Doppler gate in accordance with the position which is detected, thus enabling simple Doppler ultrasound measurement of a diseased part of the blood vessel with a high degree of reproducibility. Consequently, the ultrasound diagnostic apparatus, and the control method thereof, relating to the present invention enables reduced examination time and improved diagnostic accuracy in Doppler ultrasound measurement of a blood vessel or circulatory organ, and therefore has a high degree of applicability in the field of medical diagnostic apparatuses.

### REFERENCE SIGNS LIST

10, 20, 30 ultrasound diagnostic apparatus
101 ultrasound image acquisition unit
102 vascular region detection unit
103, 204, 301 measurement position determination unit
104 Doppler gate setting unit
105 Doppler measurement unit
106 measurement information display unit
201 3D shape analysis unit
202 position information acquisition unit
203 cross-sectional image generation unit
302 diseased part detection unit
303 measurement information display unit

What is claimed is:

1. An ultrasound diagnostic apparatus for measuring blood flow velocity by emitting ultrasound towards a measurement target via an ultrasound probe and by receiving a reflected wave via the ultrasound probe, the ultrasound diagnostic apparatus comprising:
an ultrasound image acquisition unit configured to acquire an ultrasound image which has been captured of the measurement target;
a vascular region detection unit configured to detect, from the ultrasound image, a vascular region corresponding to a blood vessel in the measurement target;
a measurement position determination unit configured to detect a specific part of the vascular region based on shape of the vascular region and to determine, in accordance with the specific part, one or more measurement positions in the vascular region in terms of longitudinal direction thereof; and
a Doppler gate setting unit configured to set, with respect to each of the measurement positions, a Doppler gate in a lumen region of the vascular region.

2. The ultrasound diagnostic apparatus of claim 1, wherein
the vascular region detection unit detects a vascular wall region of the vascular region during detection of the vascular region, and
the measurement position determination unit detects the specific part based on shape of the vascular wall region.

3. The ultrasound diagnostic apparatus of claim 2, wherein
the specific part detected by the measurement position determination unit is a part of the vascular region in which thickness of the vascular wall region exceeds a predetermined value.

4. The ultrasound diagnostic apparatus of claim 3, wherein
the measurement position determination unit determines each of the measurement positions to be in proximity to the specific part.

5. The ultrasound diagnostic apparatus of claim 1, wherein
the measurement position determination unit detects the specific part based on a change in shape of the vascular region in terms of the longitudinal direction, performs segmentation of the vascular region in accordance with the specific part, and determines a measurement position in each segment generated by the segmentation.

6. The ultrasound diagnostic apparatus of claim 5, wherein
the change in shape of the vascular region corresponds to a change in diameter of the blood vessel, a change in a vascular wall of the blood vessel, or a bifurcation of the blood vessel.

7. The ultrasound diagnostic apparatus of claim 6, wherein
the vascular region corresponds to a carotid artery in the measurement target, and
the specific part detected by the measurement position determination unit corresponds to a boundary between a common carotid artery and a carotid sinus, or to a boundary between the carotid sinus, and an internal carotid artery and an external carotid artery, in the measurement target.

8. The ultrasound diagnostic apparatus of claim 6, wherein
the measurement position determination unit determines each of the measurement positions to be in proximity to a center point of a corresponding one of the segments.

9. The ultrasound diagnostic apparatus of claim 1, wherein
the ultrasound image acquisition unit acquires the ultrasound image of the measurement target via the ultrasound probe.

10. The ultrasound diagnostic apparatus of claim 1, further comprising
a probe position detection unit configured to detect position of the ultrasound probe and orientation of a scan plane of the ultrasound probe, wherein
the ultrasound image acquisition unit acquires a plurality of ultrasound images, captured of the measurement target along different scan planes of the ultrasound probe, and generates a three dimensional image from the plurality of ultrasound images, and
the vascular region detection unit detects the vascular region from the three dimensional image.

11. The ultrasound diagnostic apparatus of claim 10, further comprising
a display unit configured to display the position of the ultrasound probe and the orientation of the scan plane which are detected, superposed on the three dimensional image, and to further display superposed information for guiding the ultrasound probe to a position for measuring blood flow velocity in accordance with the Doppler gate set by the Doppler gate setting unit.

12. The ultrasound diagnostic apparatus of claim 1, further comprising
a vascular region comparison unit, wherein
the ultrasound image acquisition unit acquires a first image and a second image that differ in terms of capture time,
the vascular region detection unit detects a first vascular region from the first image and a second vascular region from the second image,
the vascular region comparison unit compares the first vascular region to the second vascular region and judges whether a difference therebetween exceeds a predetermined threshold, and
when the vascular region comparison unit judges affirmatively, the position determination unit does not determine a measurement position and the ultrasound image acquisition unit acquires a new ultrasound image.

13. A control method for an ultrasound diagnostic apparatus for measuring blood flow velocity by emitting ultrasound towards a measurement target via an ultrasound probe and by receiving a reflected wave via the ultrasound probe, the control method comprising:
acquiring an ultrasound image which has been captured of the measurement target;
detecting, from the ultrasound image, a vascular region corresponding to a blood vessel in the measurement target;
detecting a specific part of the vascular region based on shape of the vascular region and determining, in accordance with the specific part, one or more measurement positions in the vascular region in terms of longitudinal direction thereof; and
setting, with respect to each of the measurement positions, a Doppler gate in a lumen region of the vascular region.

14. A non-transitory computer readable recording medium storing therein a program for causing a processor used in an ultrasound diagnostic apparatus to perform control processing comprising:
acquiring an ultrasound image which has been captured of a measurement target;
detecting, from the ultrasound image, a vascular region corresponding to a blood vessel in the measurement target;
detecting a specific part of the vascular region based on shape of the vascular region and determining, in accordance with the specific part, one or more measurement positions in the vascular region in terms of longitudinal direction thereof; and
setting, with respect to each of the measurement positions, a Doppler gate in a lumen region of the vascular region.
